(19) United States
(12) Patent Application Publication
(10) Pub. No.: US 2006/0019952 A1
(43) Pub. Date: Jan. 26, 2006

(54) Anti-viral therapeutics

(75) Inventors: Peter Distefano, Southboro, MA (US); Alan D. Watson, Lexington, MA (US); Rory Curtis, Ashland, MA (US); Bard J. Geesaman, Cambridge, MA (US); L. Edward Cannon, Cambridge, MA (US); Manuel A. Navia, Lexington, MA (US)

Correspondence Address: FISH & RICHARDSON PC, P.O. BOX 1022, MINNEAPOLIS, MN, 55440-1022, US

(73) Assignee: ELIXIR PHARMACEUTICALS, INC.

(21) Appl. No.: 11/048,490

(22) Filed: Jan. 31, 2005

Related U.S. Application Data
(60) Provisional application No. 60/540,444, filed on Jan. 29, 2004.

(51) Int. Cl. A61K 31/5377; A61K 31/519; A61K 31/381; A61K 31/40

(52) U.S. Cl. 514/235.5; 514/260.1; 514/447; 514/426

(57) ABSTRACT

Heterocyclic compounds of formula (I), (II), (III), and (IV) and methods of treating or preventing an HIV-mediated disorder by administering a compound of formula (I), (II), (III), or (IV) are described herein.

### CLAIM OF PRIORITY

This application claims priority under 35 USC § 119(e) to U.S. Patent Application Ser. No. 60/540,444, filed on Jan. 29, 2004, the entire contents of which are hereby incorporated by reference.

### BACKGROUND

The Sir2 protein is a deacetylase which uses NAD as a cofactor (Imai et al., 2000; Moazed, 2001; Smith et al., 2000; Tanner et al., 2000; Tanny and Moazed, 2001). Unlike other deacetylases, many of which are involved in gene silencing, Sir2 is insensitive to histone deacetylase inhibitors like trichostatin A (TSA) (Imai et al., 2000; Landry et al., 2000a; Smith et al., 2000).

Modulators of sirtuin activity would be useful in modulating various cellular processes including, e.g., repair of DNA damage, apoptosis, oncogenesis, gene silencing and senescence, inter alia.

SIRT1 deacetylates the HIV Tat protein and is required for Tat-mediated Transactivation of the HIV Promoter. (Melanie Ott, Title, Workshop 1, Molecular Mechanisms of HIV Pathogenesis, Keystone Symposia, as printed from http://www.keystonesymposia.org/MeetingsJViewMeetings.cfm?MeetingID=694 on Jan. 28, 2004.)

### SUMMARY

The invention relates to substituted heterocyclic compounds, compositions comprising the compounds, and methods of using the compounds and compound compositions. The compounds and compositions comprising them are useful for treating viral infection or viral disease or viral infection or viral disease symptoms, including AIDS. The compounds can modulate SIRT1 activity. SIRT1 deacetylates the HIV Tat protein and is required for Tat-mediated transactivation of the HIV promoter.

In one aspect, this invention relates to a method for treating or preventing a viral disorder, e.g., an infection or disease, in a subject, e.g., AIDS. The method includes administering to the subject an effective amount of a compound having a formula (I): 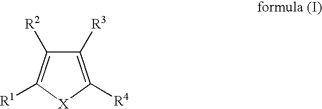
wherein;
R¹ is H, halo, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl; or when taken together with R² and the carbon to which it is attached, forms C₅-C₁₀ cycloalkenyl, C₅-C₁₀ heterocycloalkenyl, C₆-C₁₀ aryl, or C₆-C₁₀ heteroaryl; each of which can be optionally substituted with 1-5 R⁵;
R² is H, halo, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl; or when taken together with R² and the carbon to which it is attached, forms C₅-C₁₀ cycloalkenyl, C₅-C₁₀ heterocycloalkenyl, C₆-C₁₀ aryl, or C₆-C₁₀ heteroaryl; each of which can be optionally substituted with 1-5 R⁶;
each of R³ and R⁴ is, independently, H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃R⁹, sulfate, S(O)N(R⁹)₂, S(O)₂N(R⁹)₂, phosphate, C₁-C₄ alkylenedioxy, acyl, amido, aminocarbonyl, C₁-C₆ alkyl aminocarbonyl, C₁-C₆ dialkyl aminocarbonyl, aminocarbonylalkyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl; each of which is independently substituted with one or more R⁷;
each or R⁵ and R⁶ is, independently, halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, oxo, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃R⁹, sulfate, S(O)N(R⁹)₂, S(O)₂N(R⁹)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl;
each R⁷ is independently C₁-C₁₀ alkyl, C₁-C₆ haloalkyl, aminocarbonyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₇-C₁₂ heterocyclylalkyl, C₇-C₁₂ cyloalkylalkyl, C₇-C₁₂ heterocycloalkenylalkyl, or C₇-C₁₂ cycloalkenylalkyl; each of which is optionally substituted with 1-4 R¹⁰;
X is NR, O, or S;
R⁸ is H, C₁-C₆ alkyl, C₆-C₁₀ aryl, C₅-C₁₀ heteroaryl, C₇-C₁₂ arylalkyl, C₇-C₁₂ heteroarylalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₇-C₁₂ heterocyclylalkyl, C₇-C₁₂ cyloalkylalkyl, C₇-C₁₂ heterocycloalkenylalkyl, or C₇-C₁₂ cycloalkenylalkyl;
R⁹ is H or C₁-C₆ alkyl; and
each R¹⁰ is independently halo, hydroxy, alkoxy, alkyl, alkenyl, alkynl, nitro, amino, cyano, amido, or aminocarbonyl.

In some embodiments R¹ and R², taken together, with the carbons to which they are attached, form C₅-C₁₀ cycloalkenyl, C₅-C₁₀ heterocycloalkenyl, C₆-C₁₀ aryl, or C₆-C₁₀ heteroaryl.

In some embodiments R¹ and R², taken together, with the carbons to which they are attached, form C₅-C₁₀ cycloalkenyl.

In some embodiments, R¹ and R², taken together, with the carbons to which they are attached, form C₅-C₁₀ cycloalkenyl, optionally substituted with 1 or 2 C₁-C₆ alkyl.

In certain imbodiments, R¹ and R², taken together form a C₅-C₇ cycloalkenyl ring substituted with C₁-C₆ alkyl.

In certain embodiments, R¹ is C₆-C₁₀ aryl, C₅-C₁₀ heteroaryl, C₇-C₁₂ aralkyl, C₇-C₁₂ heteroaralkyl, C₃-C₈ heterocyclyl, C₅-C₁₀ cycloalkenyl, or C₅-C₁₀ heterocycloalkenyl.

In certain embodiments, R¹ is C₆-C₁₀ aryl.

In certain embodiments, R² is H, halo, C₁-C₁₀ alkyl, or C₁-C₆ haloalkyl.

In certain embodiments R³ is carboxy, cyano, aminocarbonyl, C₁-C₆ alkyl aminocarbonyl, C₁-C₆ dialkyl aminocarbonyl, C₁-C₁₀ alkoxycarbonyl, C₁-C₁₀ alkylthioylcarbonyl, hydrazinocarbonyl, C₁-C₆ alkylhydrazinocarbonyl, C₁-C₆ dialkyl hydrazinocarbonyl, or hydroxyaminocarbonyl.

In other embodiments R³ is aminocarbonyl, C₁-C₆ alkyl aminocarbonyl, C₁-C₆ dialkyl aminocarbonyl, hydrazinocarbonyl, C₁-C₆ alkyl hydrazinocarbonyl, C₁-C₆ dialkyl hydrazinocarbonyl, or hydroxyaminocarbonyl.

In other embodiments R³ is aminocarbonyl, C₁-C₆ alkyl aminocarbonyl, or C₁-C₆ dialkyl aminocarbonyl.

In certain instances R³ is H, thioalkoxy or thioaryloxy.

In still other embodiments R⁴ is nitro, amino, C₁-C₆ alkyl amino, C₁-C₆ dialkyl amino, or amido.

In still other embodiments R⁴ is amino or alteratively amido.

In some instance, R⁴ is aminocarbonylalkyl. In certain instances, the amino of the aminocarbonylalkyl is substituted, for example, with aryl, arylalkyl, alkyl, etc. In each instance, the substituent can be further substituted, for example, with halo, hydroxy, or alkoxy.

In some embodiments, R³ is aminocarbonyl, C₁-C₆ alkyl aminocarbonyl, or C₁-C₆ dialkyl aminocarbonyl; and R⁴ is amino, C₁-C₆ alkyl amino C₁-C₆ dialkyl amino or amido.

In certain embodiments X is S.

In certain embodiments X is NR⁸. In certain instances, R⁸ is H, C₁-C₆ alkyl or C₇-C₁₀ arylalkyl.

In certain embodiments
R¹ is C₆-C₁₀ aryl, C₅-C₁₀ heteroaryl, C₇-C₁₂ aralkyl, C₇-C₁₂ heteroaralkyl, C₃-C₈ heterocyclyl, C₅-C₁₀ cycloalkenyl, or C₅-C₁₀ heterocycloalkenyl; or when taken together with R² and the carbon to which it is attached, forms C₅-C₁₀ cycloalkenyl;
R² is H, halo, C₁-C₁₀ alkyl, C₁-C₆ haloalkyl; or when taken together with R¹ and the carbon to which it is attached, forms C₅-C₁₀ cycloalkenyl;
R³ is aminocarbonyl, C₁-C₆ alkyl aminocarbonyl, C₁-C₆ dialkyl aminocarbonyl, hydrazinocarbonyl, C₁-C₆ alkyl hydrazinocarbonyl, C₁-C₆ dialkyl hydrazinocarbonyl, or hydroxyaminocarbonyl;
R⁴ is amino, C₁-C₆ alkyl amino, C₁-C₆ dialkyl amino, or amido; and
X is S.

In certain embodiments
R¹ and R², taken together with the carbons to which they are attached, form C₅-C₁₀ cycloalkenyl;
R³ is aminocarbonyl, C₁-C₆ alkyl aminocarbonyl, or C₁-C₆ dialkyl aminocarbonyl;
R⁴ is amino, C₁-C₆ alkyl amino, C₁-C₆ dialkyl amino, or amido; and
X is S.

In another aspect, this invention relates to a method for treating or preventing a disorder in a subject, e.g., a disorder described herein. The method includes administering to the subject an effective amount of a compound having a formula (II): 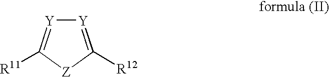
wherein;
R¹¹ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃(R¹³), sulfate, S(O)N(R¹³)₂, S(O)₂N(R¹³)₂, phosphate, C₁-C₄ alkylenedioxy, acyl, amido, aminocarbonyl, aminocarbonylalkyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl; wherein each is optionally substituted with R¹⁴;
R¹² is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃(R³), sulfate, S(O)N(R³)₂, S(O)₂N(R³)₂, phosphate, C₁-C₄ alkylenedioxy, acyl, amido, aminocarbonyl, aminocarbonylalkyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, or hydroxyaminocarbonyl or alkoxyaminocarbonyl; wherein each is optionally substituted with R¹⁵;
R¹³ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl;
R¹⁴ is hydroxy, carboxy, carboxylate, cyano, nitro, amino, C₁-C₆ alkyl amino, C₁-C₆ dialkyl amino, oxo, mercapto, thioalkoxy, thioaryloxy, thioheteroaryloxy, SO₃H, sulfate, S(O)NH₂, S(O)₂NH₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl;
R¹⁵ is halo, hydroxy, C₁-C₁₀ alkyl, C₁-C₆ haloalkyl, C₁-C₁₀ alkoxy, C₁-C₆ haloalkoxy, C₆-C₁₀ aryloxy, C₅-C₁₀ heteroaryloxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ arylalkoxy, or C₅-C₁₀ heteroarylalkoxy;
Z is NR¹⁶, O, or S;
each Y is independently N or CR¹⁸;
R¹⁶ is H, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₅-C₁₀ cycloalkenyl, C₅-C₁₀ heterocycloalkenyl, C₂-C₁₂ alkenyl, C₂-C₁₂ alkynyl; or one of R¹¹ or R¹² and R¹⁶ form a cyclic moiety containing 4-6 carbons, 1-3 nitrogens, 0-2 oxygens and 0-2 sulfurs; wherein each is optionally substituted with R¹⁷;
R¹⁷ is halo, hydroxy, C₁-C₆ alkyl, C₁-C₆ haloalkyl, C₁-C₆ alkoxy, C₁-C₆ haloalkoxy, C₂-C₈ alkenyl, C₂-C₈ alkynyl, oxo, mercapto, thioalkoxy, SO₃H, sulfate, S(O)NH₂, S(O)₂NH₂, phosphate, acyl, amido, aminocarbonyl, C₁-C₆ alkyl aminocarbonyl, C₁-C₆ dialkyl aminocarbonyl, C₁-C₆ alkoxycarbonyl, C₁-C₆ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl; and
R¹⁸ is H, halo, or C₁-C₆ alkyl.

In certain embodiments Z is NR¹⁶.

In certain embodiments Z is NR¹⁶, and R¹⁶ is C₁-C₁₀ alkyl, cycloalkenyl, C₅-C₁₀ heterocycloalkenyl, C₆-C₁₀ aryl, C₅-C₁₀ heteroaryl, C₇-C₁₂ aralkyl, or C₇-C₁₂ heteroaralkyl.

In certain embodiments R¹⁶ is C₁-C₁₀ alkyl, C₆-C₁₀ aryl, C₅-C₁₀ heteroaryl, C₇-C₁₂ aralkyl, or C₇-C₁₂ heteroaralkyl, substituted with one or more halo, alkyl, or alkoxy.

In certain embodiments R¹¹ is mercapto, thioalkoxy, thioaryloxy, thioheteroaryloxy, SO₃(R¹³), sulfate, S(O)N(R¹³)₂, S(O)₂N(R¹³)₂.

In certain embodiments R¹¹ is thioalkoxy, thioaryloxy, thioheteroaryloxy.

In certain embodiments R¹¹ is thioalkoxy, thioaryloxy, thioheteroaryloxy; substituted with one or more acyl, amido aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl.

In certain embodiments R¹¹ is thioalkoxy substituted with one or more amido, aminocarbonyl, C₁-C₆ alkyl aminocarbonyl, or C₁-C₆ dialkyl aminocarbonyl.

In certain embodiments R¹¹ is thioalkoxy substituted with aminocarbonyl.

In certain embodiments R¹² is C₁-C₁₀ alkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl.

In certain embodiments R¹² is C₁-C₁₀ alkyl, C₆-C₁₀ aryl, C₅-C₁₀ heteroaryl, C₇-C₁₂ aralkyl, or C₇-C₁₂ heteroaralkyl.

In certain embodiments R¹² is C₁-C₁₀ alkyl substituted with one or more halo, hydroxy, C₁-C₁₀ alkyl, C₁-C₆ haloalkyl, C₁-C₁₀ alkoxy, C₆-C₁₀ aryloxy, or C₅-C₁₀ heteroaryloxy.

In certain embodiments R¹² is C₁-C₁₀ alkyl substituted with aryloxy.

In some embodiments each Y is N.

In some embodiments
R¹¹ is thioalkoxy, thioaryloxy, thioheteroaryloxy; substituted with one or more acyl, amido aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl;
R¹² is C₁-C₁₀ alkyl substituted with one or more halo, hydroxy, C₁-C₁₀ alkyl, C₁-C₆ haloalkyl, C₁-C₁₀ alkoxy, C₆-C₁₀ aryloxy, or C₅-C₁₀ heteroaryloxy
Z is NR¹⁶;
each Y is N; and
R¹⁶ is C₁-C₁₀ alkyl, C₆-C₁₀ aryl, C₅-C₁₀ heteroaryl, C₇-C₁₂ aralkyl, or C₇-C₁₂ heteroaralkyl, substituted with one or more halo, alkyl, or alkoxy.

In still another aspect, this invention relates to a method for treating or preventing a disorder in a subject. The method includes administering to the subject an effective amount of a compound having a formula (III): 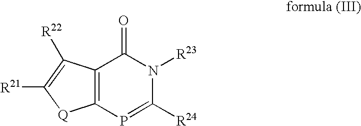
wherein;
R²¹ is halo, C₁-C₁₀ alkyl, C₁-C₆ haloalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryl, C₅-C₁₀ heteroaryl, C₇-C₁₂ aralkyl, C₇-C₁₂ heteroaralkyl; or when taken together with R²² and the carbon to which it is attached, forms C₅-C₁₀ cycloalkenyl, C₅-C₁₀ heterocycloalkenyl, C₆-C₁₀ aryl, or C₅-C₁₀ heteroaryl; each of which can be optionally substituted with 1-5 R²⁵;
R²² is halo, C₁-C₁₀ alkyl, C₁-C₆ haloalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryl, C₅-C₁₀ heteroaryl, C₇-C₁₂ aralkyl, C₇-C₁₂ heteroaralkyl; or when taken together with R²¹ and the carbon to which it is attached, forms C₅-C₁₀ cycloalkenyl, C₅-C₁₀ heterocycloalkenyl, C₆-C₁₀ aryl, or C₅-C₁₀ heteroaryl; each of which is optionally substituted with 1-5 R²⁶;
R²³ is H, halo, hydroxy, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, amino, C₁-C₆ alkyl amino, C₁-C₆ dialkyl amino, acyl, C₁-C₁₀ alkoxycarbonyl, C₁-C₁₀ thioalkoxycarbonyl;
R²⁴ is, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, amino, C₁-C₆ alkyl amino, C₁-C₆ dialkyl amino, mercapto, thioalkoxy, thioaryloxy, thioheteroaryloxy, acyl, or amidyl; each of which is optionally substituted with R²⁷;
each R²⁵ and R²⁶ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, oxo, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R²⁸)₂, S(O)₂N(R²⁸)₂, phosphate, C₁-C₄ alkylenedioxy, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl;
R²⁷ is halo, hydroxy, carboxy, carboxylate, oxo, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R²⁸)₂, S(O)₂N(R²⁸)₂, phosphate, C₁-C₄ alkylenedioxy, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl;
R²⁸ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl;
Q is S, O, or NR²⁹;
R²⁹ is H, C₁-C₆ alkyl, C₇-C₁₂ aralkyl, or C₇-C₁₂ heteroaralkyl;
P is N or CR³⁰; and
R³⁰ is H or C₁-C₆ alkyl.

In certain embodiments R²¹ and R²², together with the carbons to which they are attached, form C₅-C₁₀ cycloalkenyl, C₅-C₁₀ heterocycloalkenyl, C₆-C₁₀ aryl, or C₅-C₁₀ heteroaryl.

In certain embodiments R²¹ and R²², together with the carbons to which they are attached, form C₅-C₁₀ cycloalkenyl.

In certain embodiments R²³ is hydroxy, C₁-C₁₀ alkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, C₁-C₆ dialkyl amino, or acyl.

In certain embodiments R²³ is C₃-C₈ cycloalkyl, C₅-C₈ heterocyclyl, C₅-C₁₀ cycloalkenyl, or C₅-C₁₀ heterocycloalkenyl.

In certain embodiments R²⁴ is halo, hydroxy, C₁-C₁₀ alkyl, C₁-C₆ haloalkyl, C₁-C₁₀ alkoxy, C₁-C₆ haloalkoxy, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, C₁-C₆ alkyl amino, C₁-C₆ dialkyl amino, mercapto, thioalkoxy, thioaryloxy, or thioheteroaryloxy.

In certain embodiments R²⁴ is C₁-C₁₀ alkyl, thioalkoxy, thioaryloxy, or thioheteroaryloxy.

In certain embodiments R²⁴ is C₁-C₁₀ alkyl, thioalkoxy; and R²⁷ is carboxy, carboxylate, cyano, nitro, amino, C₁-C₆ alkyl amino, C₁-C₆ dialkyl amino, SO₃H, sulfate, S(O)N(R²⁸)₂, S(O)₂N(R²⁸)₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl.

In some embodiments R²⁴ is C₁-C₁₀ alkyl or thioalkoxy; substituted with carboxy, carboxylate, amidyl, or aminocarbonyl.

In some embodiments Q is S.

In some embodiments P is N.

In some embodiments
R²¹ and R²², together with the carbons to which they are attached, form C₅-C₁₀ cycloalkenyl, C₅-C₁₀ heterocycloalkenyl, C₆-C₁₀ aryl, or C₅-C₁₀ heteroaryl;
R²³ is hydroxy, C₁-C₁₀ alkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, C₁-C₆ dialkyl amino, or acyl;
R²⁴ is C₁-C₁₀ alkyl, thioalkoxy, thioaryloxy, or thioheteroaryloxy;
R²⁷ is carboxy, carboxylate, cyano, nitro, amino, C₁-C₆ alkyl amino, C₁-C₆ dialkyl amino, SO₃H, sulfate, S(O)N(R²⁸)₂, S(O)₂N(R²⁸)₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl;
Q is S; and
P is N.

In some embodiments
R²¹ and R²², together with the carbons to which they are attached, form C₅-C₁₀ cycloalkenyl, or C₅-C₁₀ heterocycloalkenyl;
R²³ is C₁-C₁₀ alkyl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, or C₁-C₆ dialkyl amino;
R²⁴ is C₁-C₁₀ alkyl, thioalkoxy, thioaryloxy, or thioheteroaryloxy;
R²⁷ is carboxy, carboxylate, SO₃H, sulfate, S(O)N(R²⁸)₂, S(O)₂N(R²⁸)₂, phosphate, aminocarbonyl, C₁-C₆ alkyl aminocarbonyl, C₁-C₆ dialkyl aminocarbonyl, or C₁-C₁₀ alkoxycarbonyl;
Q is S; and
P is N.

In one aspect, this invention relates to a method for treating or preventing a disorder in a subject. The method includes administering to the subject an effective amount of a compound having a formula (IV): 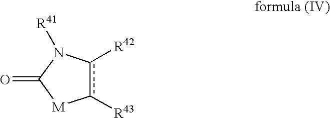
wherein;
R⁴¹ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, amino, C₁-C₆ alkyl amino, C₁-C₆ dialkyl amino, acyl, aminocarbonyl, C₁-C₆ alkyl aminocarbonyl, C₁-C₆ dialkyl aminocarbonyl, C₁-C₁₀ alkoxycarbonyl, or C₁-C₁₀ thioalkoxycarbonyl; each of which is optionally substituted with one or more R⁴⁴;
R⁴² and R⁴³, together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which is optionally substituted with 1-4 R⁴⁵; or
R⁴⁴ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R⁴⁶)₂, S(O)₂N(R⁴⁶)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, or hydroxyaminocarbonyl or alkoxyaminocarbonyl;
R⁴⁵ is halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, oxo, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R⁴⁶)₂, S(O)₂N(R⁴⁶)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl;
R⁴⁶ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl; and
M is NR⁴⁷, S, or O;
R⁴⁷ is H, halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, carboxy, carboxylate, amino, C₁-C₆ alkyl amino, C₁-C₆ dialkyl amino, acyl, aminocarbonyl, C₁-C₆ alkyl aminocarbonyl, C₁-C₆ dialkyl aminocarbonyl, or C₁-C₁₀ alkoxycarbonyl.

In certain embodiments R⁴² and R⁴³, together with the carbons to which they are attached, form C₆-C₁₀ aryl, or C₆-C₁₀ heteroaryl.

In certain embodiments R⁴² and R⁴³, together with the carbons to which they are attached, form phenyl.

In certain embodiments R⁴² and R⁴³, together with the carbons to which they are attached, form phenyl; and are substituted with halo or C₁-C₁₀ alkyl.

In certain embodiments R⁴¹ is C₁-C₁₀ alkyl; and R⁴⁴ is H, halo, C₆-C₁₀ aryl, C₅-C₁₀ heteroaryl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, acyl, amino, C₁-C₆ alkyl amino, C₁-C₆ dialkyl amino, amido, aminocarbonyl, C₁-C₆ alkyl aminocarbonyl, C₁-C₆ dialkyl aminocarbonyl, carboxy, or C₁-C₁₀ alkoxycarbonyl.

In certain embodiments M is O.

In some embodiments
R⁴¹ is C₁-C₁₀ alkyl; and R⁴⁴ is acyl, amino, C₁-C₆ alkyl amino, C₁-C₆ dialkyl amino, amido, aminocarbonyl, C₁-C₆ alkyl aminocarbonyl, C₁-C₆ dialkyl aminocarbonyl, carboxy, or C₁-C₁₀ alkoxycarbonyl;
R⁴² and R⁴³, together with the carbons to which they are attached, form C₆-C₁₀ aryl, or C₆-C₁₀ heteroaryl; and
M is O.

In some instances, a compound described herein reduces the activity of a FOXO transcription factor such as FoxO1 or FoxO3.

The amount can be effective to ameliorate at least one symptom of the viral disorder. For example, the disease or disorder can be a retroviral disorder, e.g., a lentiviral disorder, e.g., an HIV-mediated disorder such as AIDS. SIRT1 deacetylates the HIV Tat protein and is required for Tat-mediated transactivation of the HIV promoter. The method can further include administering a molecule of the invention in combination with an additional anti-viral treatment. E.g., a molecule of the invention can be administered in combination with an anti-viral agent, e.g., a protease inhibitor, e.g., a HIV protease inhibitor, a fusion inhibitor, an integrase inhibitor, or a reverse transcriptase inhibitor, (e.g., a nucleotide analog, e.g., AZT, or a non-nucleoside reverse transcriptase inhibitor). The method can include administering the compound more than once, e.g., repeatedly administering the compound. The compound can be administered in one or more boluses or continuously. The compound can be administered from without (e.g., by injection, ingestion, inhalation, etc), or from within, e.g., by an implanted device. The method can include a regimen that includes increasing or decreasing dosages of the compound. The amount can be effective to increase acetylation of a sirtuin substrate in at least some cells of the subject.

Administered “in combination with”, as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, a molecule of the invention is administered after another (first) anti-viral treatment has been administered to the patient but the first treatment did not achieve an optimal outcome or is no longer achieving an optimal outcome, e.g., the virus has become resistant to the first treatment.

The method can include administering the compound locally.

The amount can be effective to increase acetylation of a sirtuin substrate (e.g., a viral sirtuin substrate such as tat or a tat-like transactivator, or a cellular sirtuin substrate that participates in the viral lifecycle) in at least some cells of the subject.

The subject can be a mammal, e.g., a human.

The method further can include identifying a subject in need of such treatment, e.g., by evaluating sirtuin activity in a cell of the subject, evaluating nucleotide identity in a nucleic acid of the subject that encodes a sirtuin, evaluating the subject for a virus (e.g., HIV) or a virally infected cell or neoplastic cells whose growth properties are altered by a viral infection, evaluating the genetic composition or expression of genes in a cell of the subject, e.g., a virally infected cell.

The method further can include identifying a subject in need of such treatment, e.g., by evaluating by parameter such as sirtuin activity, HIV level, the level or a selected T cell or other cell surface marker, the presence of an additional infectious agents (e.g., TB) in the subject, determining if the value determined for the parameter has a predetermined relationship with a reference value, e.g., the subjects T cell count is below a threshold level, and administering the treatment to the patient.

The method can further include monitoring the subject, e.g., imaging the subject, evaluating viral load or virally infected cells in the subject, evaluating sirtuin activity in a cell of the subject, or evaluating the subject for side effects, e.g., renal function.

In one aspect, this invention relates to a method for treating or preventing a viral infection or disease or infection or disease symptoms, including AIDS in a subject. The method includes administering to the subject an effective amount of a compound depicted in Table 1, Table 2, or Table 3.

The compound can preferentially inhibit SIRT1 relative to a non-SIRT1 sirtuin, e.g., at least a 1.5, 2, 5, or 10 fold preference. The compound may preferentially inhibit another target, e.g., another sirtuin. The compound can have a Kᵢ for SIRT1 that is less than 500, 100, 50, or 40 nM.

In a further aspect, this invention relates to a method for evaluating a plurality of compounds, the method includes: a) providing library of compound that comprises a plurality of compounds, each having a formula of a compound described herein; and b) for each of a plurality of compounds from the library, and doing one or more of: i) contacting the compound to a sirtuin test protein that comprises a functional deactylase domain of a sirtuin; ii) evaluating interaction between the compound and the sirtuin test protein in the presence of the compound; and iii) evaluating ability of the compound to modulate a virus, e.g., a retrovirus, e.g., a lentivirus, e.g., HIV, e.g., in a cell.

Additional examples of embodiments are described below.

In one embodiment, evaluating the interaction between the compound and the sirtuin test protein includes evaluating enzymatic activity of the sirtuin test protein.

In one embodiment, evaluating the interaction between the compound and the sirtuin test protein includes evaluating a binding interaction between the compound and the sirtuin test protein.

The method can further include selecting, based on results of the evaluating, a compound that modulates deacetylase activity for a substrate. The substrate can be an acetylated lysine amino acid, an acetylated substrate or an acetylated peptide thereof.

The method may also further include selecting, based on results of the evaluating, a compound that modulates sirtuin deacetylase activity of a substrate.

The method may also further include selecting, based on results of the evaluating, a compound that modulates the sirtuin.

In one aspect, this invention relates to a conjugate that includes: a targeting agent and a compound, wherein the targeting agent and the compound are covalently linked, and the compound has a formula described herein.

Embodiments can include one or more of the following. The targeting agent can be an antibody, e.g., specific for a cell surface protein of a virally infected cell, e.g., a viral receptor (e.g., CD4) or a viral antigen. The targeting agent can be a synthetic peptide. The targeting agent can be a domain of a naturally occurring protein.

In another aspect, this invention relates to a kit which includes: a compound described herein, and instructions for use for treating a viral disease, viral infection, or viral disorder described herein. The kit may further include a printed material comprising a rendering of the structure of the name of the compound.

In another aspect, this invention relates to a method of analyzing or designing structures, the method includes: providing a computer-generated image or structure (preferably a three dimensional image or structure) for a compound described herein, e.g., a compound of formula I, formula II or formula III, providing a computer-generated image or structure (preferably a three dimensional image or structure) for a second compound, e.g., another compound described herein, (e.g., a compound of formula I, formula II or formula III, NAD) or a target, e.g., a sirtuin (e.g., a human sirtuin, e.g., SIRT1, SIRT2, SIRT3, SIRT4, SIRT5, SIRT6, or SIRT7) or an off-target molecule, e.g., a sirtuin other than SIRT1, e.g., SIRT2 or SIRT3, or non-sirtuin histone deacetylase; and comparing the structure of the first and second compound, e.g., a parameter related to bond angle, inter- or intra-molecular distance, position of an atom or moiety; e.g., a first or second generation compound; e.g., the predicted ability of compound to interact or inhibit a target or off-target molecule.

In a preferred embodiment, the structure is further evaluated in vitro, in vivo, or in silico with target or off-target molecule.

In a further aspect, this invention relates to a database, which includes: information about or identifying the structure, information about activity of the structure, e.g., in vitro, in vivo or in silico, e.g., at least 5, 10, 50, or 100 records.

In one aspect, this invention relates to a database, which includes a plurality of records, each record having: a) information about or identifying a compound that has a structure described herein, e.g., a structure of formula I, formula II or formula III; and b) information about a parameter of a patient, the parameter relating to a viral disorder or a patient parameter, e.g., viral load, white blood cell count, weight, etc.

In one aspect, this invention relates to a method of evaluating a compound, the method includes: providing a first compound that has a structure of a formula described herein, or a data record having information about the structure; providing a second compound that has a structure of a formula described herein or not having a formula described herein, or a data record having information about the structure; evaluating a first compound and the second compound, e.g., in vivo, in vitro, or in silico; and comparing the ability of a second compound to interact, e.g., inhibit a sirtuin, e.g., SIRT1, with a first compound, thereby evaluating ability of the second compound to interact with SIRT1.

In other aspects, the invention relates to a composition comprising a compound of any of the formulae herein, and a pharmaceutically acceptable carrier. The composition may contain an additional therapeutic agent (for example one, two, three, or more additional agents), e.g., an anti-viral agent, e.g., a protease inhibitor, e.g., a HIV protease inhibitor, a fusion inhibitor, an integrase inhibitor, and/or a reverse transcriptase inhibitor, (e.g., a nucleotide analog, e.g., AZT, or a non-nucleoside reverse transcriptase inhibitor). Also within the scope of this invention is the use of such a composition for the manufacture of a medicament for anti-viral use.

In another aspect, the invention is a method for treating or preventing a viral disease, e.g., HIV, in a subject. The method includes administering a SIRT1 antagonist described herein, e.g., having a structure of formula (I).

In another aspect, the invention includes a method for treating or preventing a tat or tat mediated disease or disorder. The method includes administering a compound described herein, e.g., a compound of formula (I).

In one embodiment, the method includes administering a SIRT1 antagonist in combination with one or more therapeutic agents, e.g., a therapeutic agent or agent for treating a viral disorder, e.g., a viral disorder described herein. The additional agents may be administered in a single composition with the SIRT1 antagonist or may be administered separately, for example in separate formulations such as separate pills. When administered in separate formulations, the agents can be administered at the same time, or at different times. Exemplary additional agents include a protease inhibitor, e.g., a HIV protease inhibitor, a fusion inhibitor, an integrase inhibitor, or a reverse transcriptase inhibitor, (e.g., a nucleotide analog, e.g., AZT, or a non-nucleoside reverse transcriptase inhibitor). Specific examples include saquinavir, ritonavir, indinavir, nelfinavir, saquinavir, amprenavir, lopinavir, emtricitabine, tenofovir disoproxil fumarate, and combinations thereof, e.g., a fixed-dose combination of emtricitabine and tenofovir disoproxil fumarate.

The SIRT1 antagonist and the therapeutic agents can be administered simultaneously or sequentially.

Also within the scope of this invention is a packaged product. The packaged product includes a container, one of the aforementioned compounds in the container, and a legend (e.g., a label or insert) associated with the container and indicating administration of the compound for treating a viral disease, a viral disorder, or viral infection described herein.

The subject can be a mammal, preferably a human. The subject can also be a non-human subject, e.g., an animal model. In certain embodiments the method can further include identifying a subject. Identifying a subject in need of such treatment can be in the judgment of a subject or a health care professional and can be subjective (e.g., opinion) or objective (e.g., measurable by a test or diagnostic method).

The term “mammal” includes organisms, which include mice, rats, cows, sheep, pigs, rabbits, goats, and horses, monkeys, dogs, cats, and preferably humans.

The term “treating” or “treated” refers to administering a compound described herein to a subject with the purpose to cure, heal, alleviate, relieve, alter, remedy, ameliorate, improve, or affect a disease, e.g., an infection, the symptoms of the disease or the predisposition toward the disease.

An effective amount of the compound described above may range from about 0.1 mg/Kg to about 500 mg/Kg, alternatively from about 1 to about 50 mg/Kg. Effective doses will also vary depending on route of administration, as well as the possibility of co-usage with other agents.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂—, —CH₂CH₂—, and —CH₂CH₂CH₂—.

The term “alkenyl” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The terms “alkylamino” and “dialkylamino” refer to —NH(alkyl) and —NH(alkyl)₂ radicals respectively. The term “aralkylamino” refers to a —NH(aralkyl) radical. The term alkylaminoalkyl refers to a (alkyl)NH-alkyl- radical; the term dialkylaminoalkyl refers to a (alkyl)₂N-alkyl- radical The term “alkoxy” refers to an —O-alkyl radical. The term “mercapto” refers to an SH radical. The term “thioalkoxy” refers to an —S-alkyl radical. The term thioaryloxy refers to an —S-aryl radical.

The term “aryl” refers to an aromatic monocyclic, bicyclic, or tricyclic hydrocarbon ring system, wherein any ring atom capable of substitution can be substituted (e.g., by one or more substituents). Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons. Any ring atom can be substituted (e.g., by one or more substituents). The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3,1-6, or 1-9 heteroatoms of N, O, or S if monocyclic, bicyclic, or tricyclic, respectively). The heteroatom may optionally be the point of attachment of the heterocyclyl substituent. Any ring atom can be substituted (e.g., by one or more substituents). The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted (e.g., by one or more substituents). The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted (e.g., by one or more substituents). The heterocycloalkenyl groups can contain fused rings. Fused rings are rings that share a common carbon atom. Examples of heterocycloalkenyl include but are not limited to tetrahydropyridyl and dihydropyranyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Any ring atom can be substituted (e.g., by one or more substituents).

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

The terms “aminocarbonyl,” alkoxycarbonyl,” hydrazinocarbonyl, and hydroxyaminocarbonyl refer to the radicals —C(O)NH₂, —C(O)O(alkyl), —C(O)NH₂NH₂, and —C(O)NH₂NH₂, respectively.

The term “amindo”refers to a —NHC(O)— radical, wherein N is the point of attachment.

The term “substituent” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

A “retroviral disorder” refers to a disorder caused at least in part by a retrovirus. In one embodiment, the retrovirus can be integrated in a cell, e.g., as a latent or newly integrated virus. In the case of latent virus, in one example, a subject having the disorder may not have a detectable viral load. In another example, the subject has a detectable, e.g., substantial, viral load.

A “lentiviral disorder” refers to a disorder caused at least in part by a lentivirus. Lentiviruses typically are infectious viruses that have 4 main genes coding for the virion proteins in the order: 5′-gag-pro-pol-env-3′. There may be additional genes depending on the virus (e.g., for HIV-1: vif, vpr, vpu, tat, rev, nef) whose products are involved in regulation of synthesis and processing virus RNA and other replicative functions. For some lentiviruses, the LRT is about 600 nt long, of which the U3 region is 450, the R sequence 100 and the U5 region some 70 nt long. Exemplary Lentiviruses include primate lentiviruses (e.g., SIV, HIV-1, HIV-2), equine lentiviruses (e.g., equine infectious anemia virus), bovine lentiviruses (e.g., bovine immunodeficiency virus), feline lentiviruses (e.g., feline immunodeficiency virus (Petuluma)), and ovine/caprine lentiviruses (e.g., arthritis encephalitis virus; 61.0.6.4.002 visna/maedi virus (strain 1514)).

In another embodiment, the retrovirus is in the form of infectious particles. For example, a subject having the disorder may have a detectable (e.g., a significant) viral load.

An exemplary “retroviral disorder” is an HIV-related disorder. An “HIV-related disorder” refers to any disorder caused at least in part by an HIV-related retrovirus, including HIV-1, HIV-2, FLV, HTLV-1, HTLV-2, and SIV. See, e.g., Coffin (1992) Curr Top Microbiol Immunol. 1992; 176:143-64 Such disorders include AIDS and AIDS-related complex (ARC), and a variety of disorders that arise as a consequence of HIV infection, e.g., Kaposi's sarcoma, non-Hodgkin's lymphomas, central nervous system non-Hodgkin's lymphomas, and rare tumors (e.g., intracranial tumors such as glioblastomas, anaplastic astrocytomas, and subependymomas), opportunistic infections (e.g., Histoplasmosis, CMV (Cytomegalovirus), Cryptosporidiosis, Cryptococcal Meningitis, Dementia and Central Nervous System Problems, Hepatitis and HIV, Hepatitis C and HIV, HPV, KS (Kaposi's Sarcoma), Lymphoma, MAC (Mycobacterium Avium Complex), Molluscum, PCP (Pneumocystis Carinii Pneumonia), PML (Progressive Multifocal Leucoencephalopathy), Shingles (Herpes Zoster), TB (Tuberculosis), Thrush (Candidiasis), Toxoplasmosis), fatigue, anemia, cachexia, and AIDS wasting.

A “viral neoplastic disorder” is a disease or disorder characterized by cells that have the capacity for autonomous growth or replication due to a virus, e.g., a viral infection. As a result the cells are in an abnormal state or condition characterized by proliferative cell growth.

Methods and compositions disclosed herein can be used to treat any viral disorder which is dependent on the state of acetylation of a protein, e.g., a viral or cellular protein involved in propagation of the virus, e.g., a viral transcription factor. Exemplary viral disorders include retroviral and lentiviral disorders.

The details of one or more embodiments of the invention are set forth in the accompanying drawings and the description below. Other features, objects, and advantages of the invention will be apparent from the description and drawings, and from the claims.

All references cited herein, whether in print, electronic, computer readable storage media or other form, are expressly incorporated by reference in their entirety, including but not limited to, abstracts, articles, journals, publications, texts, treatises, internet web sites, databases, patents, patent applications and patent publications. This application also incorporates by reference a U.S. application, titled “TREATING A VIRAL DISORDER,” filed 31 Jan. 2005, naming DiStefano et al, and assigned attorney docket number 13407-051001.

### DETAILED DESCRIPTION

Structure of Exemplary Compounds

Exemplary compounds that can be used (e.g., in a method described herein) have a general formula (I), (II), (III), or (IV) and contain a substituted cyclic (e.g., pentacyclic or hexacyclic) or polycyclic core containing one or more oxygen, nitrogen, or sulfur atoms as a constituent atom of the ring(s).

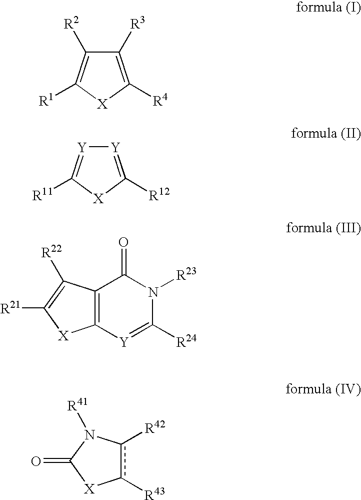

Any ring carbon atom can be substituted. The cyclic or polycyclic core may be partially or fully saturated, i.e. one or two double bonds respectively.

A preferred subset of compounds of formula (I) includes those having a ring that is fused to the pentacyclic core, e.g., R¹ and R², together with the carbons to which they are attached, and/or R³ and R⁴, together with the carbons to which they are attached, form C₅-C₁₀ cycloalkenyl (e.g., C5, C6, or C7), C₅-C₁₀ heterocycloalkenyl (e.g., C5, C6, or C7), C₆-C₁₀ aryl (e.g., C6, C8 or C10), or C₆-C₁₀ heteroaryl (e.g., C5 or C6). Fused ring combinations may include without limitation one or more of the following:

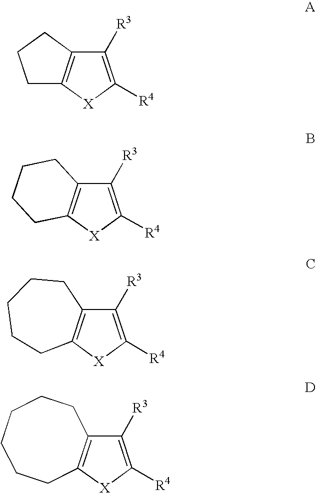

Each of these fused ring systems may be optionally substituted with substitutents, which may include without limitation halo, hydroxy, C₁-C₁₀ alkyl (C1,C2,C3,C4,C5,C6,C7,C8,C9,C10), C₁-C₆ haloalkyl (C1,C2,C3,C4,C5,C6,), C₁-C₁₀ alkoxy (C1,C2,C3,C4,C5,C6,C7,C8,C9,C10), C₁-C₆ haloalkoxy (C1,C2,C3,C4,C5,C6,), C₆-C₁₀ aryl (C6,C7,C8,C9,C10), C₅-C₁₀ heteroaryl (C5,C6,C7,C8,C9,C10), C₇-C₁₂ aralkyl (C7,C8,C9,C10,C11,C12), C₇-C₁₂ heteroaralkyl (C7,C8,C9,C10,C11,C12), C₃-C₈ heterocyclyl (C3,C4,C5,C6,C7,C8), C₂-C₁₂ alkenyl (C2,C3,C4,C5,C6,C7,C8,C9,C10,C11,C12), C₂-C₁₂ alkynyl (C2,C3,C4,C5,C6,C7,C8,C9,C10,C11,C12), C₅-C₁₀ cycloalkenyl (C5,C6,C7,C8,C9,C10), C₅-C₁₀ heterocycloalkenyl (C5,C6,C7,C8,C9,C10), carboxy, carboxylate, cyano, nitro, amino, C₁-C₆ alkyl amino (C1,C2,C3,C4,C5,C6,), C₁-C₆ dialkyl amino (C1,C2,C3,C4,C5,C6,), mercapto, SO₃H, sulfate, S(O)NH₂, S(O)₂NH₂, phosphate, C₁-C₄ alkylenedioxy (C1,C2,C3,C4), oxo, acyl, aminocarbonyl, C₁-C₆ alkyl aminocarbonyl (C1,C2,C3,C4,C5,C6,), C₁-C₆ dialkyl aminocarbonyl (C1,C2,C3,C4,C5,C6,), C₁-C₁₀ alkoxycarbonyl (C1,C2,C3,C4,C5,C6,C7,C8,C9,C10), C₁-C₁₀ thioalkoxycarbonyl (C1,C2,C3,C4,C5,C6,C7,C8,C9,C10), hydrazinocarbonyl, C₁-C₆ alkyl hydrazinocarbonyl (C1,C2,C3,C4,C5,C6,), C₁-C₆ dialkyl hydrazinocarbonyl (C1,C2,C3,C4,C5,C6,), hydroxyaminocarbonyl, etc. Preferred substituents include C₁-C₁₀ alkyl (e.g., C1, C2, C3, C4, C5, C6, C7, C8, C9, C10), aminocarbonyl, and amido. The substitution pattern can be selected as desired.

Another preferred subset of compounds of formula (I) includes those where R¹ and R² are C₁-C₆ alkyl (e.g., wherein R¹ and R² are both CH₃).

In still another preferred subset of the compounds of formula (I), R³ is a substituted or unsubstitued aminocarbonyl and R⁴ is an amido substituted with a substituent.

In still another preferred subset of the compounds of formula (I), X is S.

A preferred subset of compounds of formula (II) includes those having a triazole core (i.e., wherein X is NR¹⁶ and both Ys are N).

Another preferred subset of compounds include those where R¹¹ is a substituted thioalkoxy. Where R¹¹ is thioalkoxy, preferred substituents include aminocarbonyl. An example of a preferred subset is provided below.

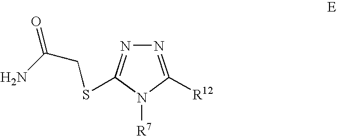

Still another subset of preferred embodiments include those where R¹² is aryl, arylalkyl, heteroaryl, heteroarylalkyl, and alky substituted with heteroaryloxy or aryloxy. Each aryl and heteroaryl is optionally substituted.

Still another subset of preferred embodiments include those wherein X is NR⁷ and R⁷ is aryl, heteroaryl, arylalkyl or heteroarylalkyl, each is which is optionally substituted.

A preferred subset of compounds of formula (III) includes those having one of the following polycyclic cores:

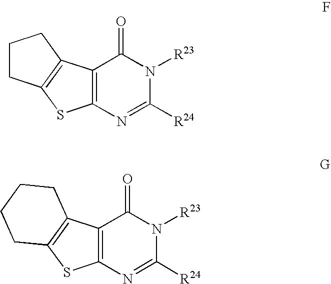

The polycyclic core can be substituted with one or more suitable substituents.

A preferred subset of compounds of formula (IV) includes those having the following polycyclic core:

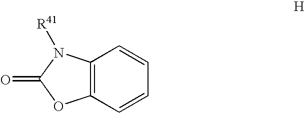

The polycyclic core can be substituted with one or more suitable substituents.

Other examples of embodiments are depicted in the following structures below together with representative examples of Sir2 activity.

TABLE 1
Activity of Triazoles (conc. in μM)

| Compound | | SirT1 | SirT2 |
|---|---|---|---|
| Number | Chemical Name | (μM) | (μM) |
| 1 | 2-[4-Benzyl-5-(1H-indol-3-ylmethyl)-4H- | B | C |
| | [1,2,4]triazol-3-ylsulfanyl]-acetamide | | |
| 2 | 2-[4-(4-Methoxy-phenyl)-5-(naphthalen-1- | B | C |
| | yloxymethyl)-4H-[1,2,4]triazol-3-ylsulfanyl]- | | |
| | acetamide | | |
| 3 | 2-(5-Benzyl-4-p-tolyl-4H-[1,2,4]triazol-3- | B | C |
| | ylsulfanyl)-acetamide | | |
| 4 | 2-[5-(2-Bromo-phenyl)-4-p-tolyl-4H- | C | B |
| | [1,2,4]triazol-3-ylsulfanyl]-acetamide | | |

TABLE 2
Activity of representative compounds (conc. in μM)

| Compound | | SirT1 | SirT2 |
|---|---|---|---|
| Number | Chemical Name | (μM) | (μM) |
| 5 | (5 -Cyclohexyl-4-oxo-2,3,4,5- | B | C |
| | tetrahydro-1H-8-thia-5,7-diaza- | | |
| | cyclopenta[a]inden-6-ylsulfanyl)-acetic acid | | |
| 6 | 2-(6-Bromo-2-oxo-benzooxazol-3- | B | C |
| | yl)-acetamide | | |
| 7 | 3-(3-Amino-4-oxo-3,4,5,6,7,8- | C | C |
| | hexahydro-benzo[4,5]thieno[2,3- | | |
| | d]pyrimidin-2-yl)-propionic acid | | |

TABLE 3
Activity of representative compounds

| Com- | | SirT1 |
|---|---|---|
| pound | | p53-382- |
| Number | Chemical Name | FdL IC50 |
| 8 | 3-Chloro-benzo[b]thiophene-2-carboxylic acid | D |
| | carbamoylmethyl ester | |
| 9 | 4,5-Dimethyl-2-[2-(5-methyl-3-nitro-pyrazol-1-yl)- | C |
| | acetylamino]-thiophene-3-carboxylic acid amide | |
| 10 | Furan-2-carboxylic acid (3-carbamoyl-4,5,6,7- | D |
| | tetrahydro-benzo[b]thiophen-2-yl)-amide | |
| 11 | 5-Bromo-furan-2-carboxylic acid (3-carbamoyl-4,5- | C |
| | dimethyl-thiophen-2-yl)-amide | |
| 12 | 2-[(Thiophene-2-carbonyl)-amino]-4,5,6,7- | D |
| | tetrahydro-benzo[b]thiophene-3-carboxylic acid | |
| | amide | |
| 13 | Furan-2-carboxylic acid (3-carbamoyl-5,6-dihydro- | D |
| | 4H-cyclopenta[b]thiophen-2-yl)-amide | |
| 14 | Tetrahydro-furan-2-carboxylic acid (3-carbamoyl-6- | D |
| | methyl-4,5,6,7-tetrahydro-benzo[b]thiophen-2-yl)- | |
| | amide | |
| 15 | Tetrahydro-furan-2-carboxylic acid (3-carbamoyl- | C |
| | 4,5-dimethyl-thiophen-2-yl)-amide | |
| 16 | 2-(3,4-Dichloro-benzoylamino)-6-methyl-4,5,6,7- | D |
| | tetrahydro-benzo[b]thiophene-3-carboxylic acid | |
| | amide | |
| 17 | 2-[2-(3-Nitro-[1,2,4]triazol-1-yl)-acetylamino]- | D |
| | 4,5,6,7-tetrahydro-benzo[b]thiophene-3-carboxylic | |
| | acid amide | |
| 18 | 2-(4-Fluoro-benzoylamino)-4,5-dimethyl-thiophene- | D |
| | 3-carboxylic acid amide | |
| 19 | 2-(3-Chloro-benzoylamino)-4,5,6,7-tetrahydro- | D |
| | benzo[b]thiophene-3-carboxylic acid amide | |
| 20 | Pyrazine-2-carboxylic acid (3-carbamoyl-4,5,6,7- | D |
| | tetrahydro-benzo[b]thiophen-2-yl)-amide | |
| 21 | 3-Chloro-benzo[b]thiophene-2-carboxylic acid (3- | D |
| | carbamoyl-4,5-dimethyl-thiophen-2-yl)-amide | |
| 22 | 5-Bromo-N-(3-carbamoyl-4,5,6,7-tetrahydro- | D |
| | benzo[b]thiophen-2-yl)-nicotinamide | |
| 23 | 4-Bromo-1-methyl-1H-pyrazole-3-carboxylic acid | D |
| | (3-carbamoyl-5,6-dihydro-4H-cyclopenta[b]thio- | |
| | phen-2-yl)-amide | |
| 24 | 5-Bromo-furan-2-carboxylic acid (3-carbamoyl- | D |
| | 4,5,6,7-tetrahydro-benzo[b]thiophen-2-yl)-amide | |
| 25 | 2-(3,4-Dichloro-benzoylamino)-4,5,6,7-tetrahydro- | D |
| | benzo[b]thiophene-3-carboxylic acid amide | |
| 26 | 2-(Cyclopropanecarbonyl-amino)-4,5-dimethyl-C | |
| | thiophene-3-carboxylic acid amide | |
| 27 | 2-(Cyclohexanecarbonyl-amino)-4,5,6,7-tetrahydro- | D |
| | benzo[b]thiophene-3-carboxylic acid amide | |
| 28 | 2-(2,5-Dichloro-benzoylamino)-4,5-dimethyl- | D |
| | thiophene-3-carboxylic acid amide | |
| 29 | N-(3-Carbamoyl-4,5-dimethyl-thiophen-2-yl)- | C |
| | isonicotinamide | |
| 30 | Pyrazine-2-carboxylic acid (3-carbamoyl-4,5- | C |
| | dimethyl-thiophen-2-yl)-amide | |
| 31 | 2-(5-Pyridin-4-yl-2H-[1,2,4]triazol-3-yl)-acetamide | D |
| 32 | 2-(Cyclopentanecarbonyl-amino)-6-methyl-4,5,6,7- | A |
| | tetrahydro-benzo[b]thiophene-3-carboxylic acid | |
| | amide | |
| 33 | 2-(3-Methyl-butyrylamino)-4,5,6,7,8,9-hexahydro- | C |
| | cycloocta[b]thiophene-3-carboxylic acid amide | |
| 34 | 2-(Cyclopropanecarbonyl-amino)-5,6,7,8-tetrahydro- | C |
| | 4H-cyclohepta[b]thiophene-3-carboxylic acid amide | |
| 35 | 6-Methyl-2-propionylamino-4,5,6,7-tetrahydro- | B |
| | benzo[b]thiophene-3-carboxylic acid amide | |
| 36 | 2-Amino-6-methyl-4,5,6,7-tetrahydro- | C |
| | benzo[b thiophene-3-carboxylic acid amide | |
| 37 | 2-Amino-5-phenyl-thiophene-3-carboxylic acid | C |
| | amide | |
| 38 | 2-Amino-6-ethyl-4,5,6,7-tetrahydro- | C |
| | benzo[b]thiophene-3-carboxylic acid amide | |
| 39 | 2-(1-Benzyl-3-methylsulfanyl-1H-indol-2-yl)-N-p- | D |
| | tolyl-acetamide | |
| 40 | N-Benzyl-2-(1-methyl-3-phenylsulfanyl-1H-indol-2- | D |
| | yl)-acetamide | |
| 41 | N-(4-Chloro-phenyl)-2-(1-methyl-3-phenylsulfanyl- | D |
| | 1H-indol-2-yl)-acetamide | |
| 42 | N-(3-Hydroxy-propyl)-2-(1-methyl-3-phenyl- | D |
| | sulfanyl-1H-indol-2-yl)-acetamide | |
| 43 | 2-(1-Benzyl-3-phenylsulfanyl-1H-indol-2-yl)-N-(3- | D |
| | hydroxy-propyl)-acetamide | |
| 44 | 2-(1-Benzyl-3-methylsulfanyl-1H-indol-2-yl)-N-(4- | D |
| | methoxy-phenyl)-acetamide | |
| 45 | 2-(1-Benzyl-1H-indol-2-yl)-N-(4-methoxy-phenyl)- | D |
| | acetamide | |
| 46 | 2-(1-Methyl-3-methylsulfanyl-1H-indol-2-yl)-N-p- | D |
| | tolyl-acetamide | |
| 47 | 2-(1-Benzyl-3-methylsulfanyl-1H-indol-2-yl)-N-(2- | D |
| | chloro-phenyl)-acetamide | |
| 48 | 2-(1,5-Dimethyl-3-methylsulfanyl-1H-indol-2-yl)-N- | C |
| | (2-hydroxy-ethyl)-acetamide | |
| 49 | 2-(1-Benzyl-1H-indol-2-yl)-N-(2-chloro-phenyl)- | D |
| | acetamide | |

* Compounds having activity designated with an A have an IC₅₀ of less than 1.0 μM. Compounds having activity designated with a B have an IC₅₀ between 1.0 μM and 10.0 μM. Compounds having activity designated with a C have an IC₅₀ greater than 10.0 μM. Compounds designated with a D were not tested in this assay.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Compounds that can be useful in practicing this invention can be identified through both in vitro (cell and non-cell based) and in vivo methods. A description of these methods is described in the Examples.

Synthesis of Compounds

In many instances, the compounds described herein, or precursors thereof, can be purchased commercially, for example from Asinex, Moscow, Russia; Bionet, Camelford, England; ChemDiv, San Diego, Calif.; Comgenex, Budapest, Hungary; Enamine, Kiev, Ukraine; IF Lab, Ukraine; Interbioscreen, Moscow, Russia; Maybridge, Tintagel, UK; Specs, The Netherlands; Timtec, Newark, Del.; Vitas-M Lab, Moscow, Russia.

Alternatively, the compounds described herein can be synthesized by conventional methods. As can be appreciated by the skilled artisan, methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein can be separated from a reaction mixture and further purified by methods such as column chromatography, high-pressure liquid chromatography, or recrystallization. Techniques useful for the separation of isomers, e.g., stereoisomers are within skill of the art and are described in Eliel, E. L.; Wilen, S. H.; Mander, L. N. Stereochemistry of Organic Compounds, Wiley Interscience, NY, 1994.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

The compounds of this invention may be modified by appending appropriate functionalities to enhance selected biological properties, e.g., targeting to a particular tissue. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60). Thus, one embodiment relates to a method of using the compounds described herein for generating derivatives or chemical libraries comprising: 1) providing a body comprising a plurality of wells; 2) providing one or more compounds identified by methods described herein in each well; 3) providing an additional one or more chemicals in each well; 4) isolating the resulting one or more products from each well. An alternate embodiment relates to a method of using the compounds described herein for generating derivatives or chemical libraries comprising: 1) providing one or more compounds described herein attached to a solid support; 2) treating the one or more compounds identified by methods described herein attached to a solid support with one or more additional chemicals; 3) isolating the resulting one or more products from the solid support. In the methods described above, “tags” or identifier or labeling moieties may be attached to and/or detached from the compounds described herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

Sirtuins

Sirtuins are members of the Silent Information Regulator (SIR) family of genes. Sirtuins are proteins that include a SIR2 domain as defined as amino acids sequences that are scored as hits in the Pfam family “SIR2”-PF02146. This family is referenced in the INTERPRO database as INTERPRO description (entry IPR003000). To identify the presence of a “SIR2” domain in a protein sequence, and make the determination that a polypeptide or protein of interest has a particular profile, the amino acid sequence of the protein can be searched against the Pfam database of HMMs (e.g., the Pfam database, release 9) using the default parameters (http://www.sanger.ac.uk/Software/Pfam/HMM_search). The SIR2 domain is indexed in Pfam as PF02146 and in INTERPRO as INTERPRO description (entry IPR003000). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in “The Pfam Protein Families Database” Bateman A, Birney E, Cerruti L, Durbin R, Etwiller L, Eddy S R, Griffiths-Jones S, Howe K L, Marshall M, Sonnhammer E L (2002) Nucleic Acids Research 30(1):276-280 and Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314.

The proteins encoded by members of the SIR2 gene family may show high sequence conservation in a 250 amino acid core domain. A well-characterized gene in this family is S. cerevisiae SIR2, which is involved in silencing HM loci that contain information specifying yeast mating type, telomere position effects and cell aging (Guarente, 1999; Kaeberlein et al., 1999; Shore, 2000). The yeast Sir2 protein belongs to a family of histone deacetylases (reviewed in Guarente, 2000; Shore, 2000). The Sir2 protein is a deacetylase which can use NAD as a cofactor (Imai et al., 2000; Moazed, 2001; Smith et al., 2000; Tanner et al., 2000; Tanny and Moazed, 2001). Unlike other deacetylases, many of which are involved in gene silencing, Sir2 is relatively insensitive to histone deacetylase inhibitors like trichostatin A (TSA) (Imai et al., 2000; Landry et al., 2000a; Smith et al., 2000). Mammalian Sir2 homologs, such as SIRT1, have NAD-dependent deacetylase activity (Imai et al., 2000; Smith et al., 2000).

Exemplary mammalian sirtuins include SIRT1, SIRT2, and SIRT3, e.g., human SIRT1, SIRT2, and SIRT3. A compound described herein may inhibit one or more activities of a mammalian sirtuin, e.g., SIRT1, SIRT2, or SIRT3, e.g., with a Kᵢ of less than 500, 200, 100, 50, or 40 nM. For example, the compound may inhibit deacetylase activity, e.g., with respect to a natural or artificial substrate, e.g., a substrate described herein, e.g., as follows.

Natural substrates for SIRT1 include histones and p53. SIRT1 proteins bind to a number of other proteins, referred to as “SIRT1 binding partners.” For example, SIRT1 binds to p53 and plays a role in the p53 pathway, e.g., K370, K371, K372, K381, and/or K382 of p53 or a peptide that include one or more of these lysines. For example, the peptide can be between 5 and 15 amino acids in length. SIRT1 proteins can also deacetylate histones. For example, SIRT1 can deacetylate lysines 9 or 14 of histone H3 or small peptides that include one or more of these lysines. Histone deacetylation alters local chromatin structure and consequently can regulate the transcription of a gene in that vicinity. Many of the SIRT1 binding partners are transcription factors, e.g., proteins that recognize specific DNA sites. Interaction between SIRT1 and SIRT1 binding partners can deliver SIRT1 to specific regions of a genome and can result in a local manifestation of substrates, e.g., histones and transcription factors localized to the specific region.

Natural substrates for SIRT2 include tubulin, e.g., alpha-tubulin. See, e.g., North et al. Mol Cell. 2003 February; 11(2):437-44. Exemplary substrates include a peptide that includes lysine 40 of alpha-tubulin.

Still other exemplary sirtuin substrates include cytochrome c and acetylated peptides thereof, and HIV tat and acetylated peptides thereof.

The terms “SIRT1 protein” and “SIRT1 polypeptide” are used interchangeably herein and refer a polypeptide that is at least 25% identical to the 250 amino acid conserved SIRT1 catalytic domain, amino acid residues 258 to 451 of SEQ ID NO:1. SEQ ID NO:1 depicts the amino acid sequence of human SIRT1. In preferred embodiments, a SIRT1 polypeptide can be at least 30, 40, 50, 60, 70, 80, 85, 90, 95, 99% homologous to SEQ ID NO:1 or to the amino acid sequence between amino acid residues 258 and 451 of SEQ ID NO:1. In other embodiments, the SIRT1 polypeptide can be a fragment, e.g., a fragment of SIRT1 capable of one or more of: deacetylating a substrate in the presence of NAD and/or a NAD analog and capable of binding a target protein, e.g., a transcription factor. Such functions can be evaluated, e.g., by the methods described herein. In other embodiments, the SIRT1 polypeptide can be a “full length” SIRT1 polypeptide. The term “full length” as used herein refers to a polypeptide that has at least the length of a naturally-occurring SIRT1 polypeptide (or other protein described herein). A “full length” SIRT1 polypeptide or a fragment thereof can also include other sequences, e.g., a purification tag., or other attached compounds, e.g., an attached fluorophore, or cofactor. The term “SIRT1 polypeptides” can also include sequences or variants that include one or more substitutions, e.g., between one and ten substitutions, with respect to a naturally occurring Sir2 family member. A “SIRT1 activity” refers to one or more activity of SIRT1, e.g., deacetylation of a substrate (e.g., an amino acid, a peptide, or a protein), e.g., transcription factors (e.g., p53) or histone proteins, (e.g., in the presence of a cofactor such as NAD and/or an NAD analog) and binding to a target, e.g., a target protein, e.g., a transcription factor.

As used herein, a “biologically active portion” or a “functional domain” of a protein includes a fragment of a protein of interest which participates in an interaction, e.g., an intramolecular or an inter-molecular interaction, e.g., a binding or catalytic interaction. An inter-molecular interaction can be a specific binding interaction or an enzymatic interaction (e.g., the interaction can be transient and a covalent bond is formed or broken). An inter-molecular interaction can be between the protein and another protein, between the protein and another compound, or between a first molecule and a second molecule of the protein (e.g., a dimerization interaction). Biologically active portions/functional domains of a protein include peptides comprising amino acid sequences sufficiently homologous to or derived from the amino acid sequence of the protein which include fewer amino acids than the full length, natural protein, and exhibit at least one activity of the natural protein. Biological active portions/functional domains can be identified by a variety of techniques including truncation analysis, site-directed mutagenesis, and proteolysis. Mutants or proteolytic fragments can be assayed for activity by an appropriate biochemical or biological (e.g., genetic) assay. In some embodiments, a functional domain is independently folded. Typically, biologically active portions comprise a domain or motif with at least one activity of a protein, e.g., SIRT1. An exemplary domain is the SIRT1 core catalytic domain. A biologically active portion/functional domain of a protein can be a polypeptide which is, for example, 10, 25, 50, 100, 200 or more amino acids in length. Biologically active portions/functional domain of a protein can be used as targets for developing agents which modulate SIRT1.

The following are exemplary SIR sequences:

>sp|Q96EB6|SIR1_HUMAN NAD-dependent deacetylase
sirtuin 1 (EC 3.5.1.-) (hSIRT1) (hSIR2) (SIR2-like
protein 1) - Homo sapiens (Human).

(SEQ ID NO: 1)

MADEAALALQPGGSPSAAGADREAASSPAGEPLRKRPRRDGPGLERSPGE
PGGAAPEREVPAAARGCPGAAAAALWREAEAEAAAAGGEQEAQATAAAGE
GDNGPGLQGPSREPPLADNLYDEDDDDEGEEEEEAAAAAIGYRDNLLFGD
EIITNGFHSCESDEEDRASHASSSDWTPRPRIGPYTFVQQHLMIGTDPRT
ILKDLLPETIPPPELDDMTLWQIVINILSEPPKRKKRKDINTIEDAVKLL
QECKKIIVLTGAGVSVSCGIPDFRSRDGIYARLAVDFPDLPDPQAMFDIE
YFRKDPRPFFKFAKEIYPGQFQPSLCHKFIALSDKEGKLLRNYTQNIDTL
EQVAGIQRIIQCHGSFATASCLICKYKVDCEAVRGDIFNQVVPRCPRCPA
DEPLAIMKPEIVFFGENLPEQFHRAMKYDKDEVDLLIVIGSSLKVRPVAL
IPSSIPHEVPQILINREPLPHLHFDVELLGDCDVIINELCHRLGGEYAKL
CCNPVKLSEITEKPPRTQKELAYLSELPPTPLHVSEDSSSPERTSPPDSS
VIVTLLDQAAKSNDDLDVSESKGCMEEKPQEVQTSRNVESIAEQMENPDL
KNVGSSTGEKNERTSVAGTVRKCWPNRVAKEQISRRLDGNQYLFLPPNRY
IFHGAEVYSDSEDDVLSSSSCGSNSDSGTCQSPSLEEPMEDESEIEEFYN
GLEDEPDVPERAGGAGFGTDGDDQEAINEAISVKQEVTDMNYPSNKS
>sp|Q8IXJG|SIR2_HUMAN NAD-dependent deacetylase
sirtuin 2 (EC 3.5.1.-) (SIR2-like) (SIR2- like
protein 2) - Homo sapiens (Human).

(SEQ ID NO:2)

MAEPDPSHPLETQAGKVQEAQDSDSDSEGGAAGGEADMDFLRNLFSQTLS
LGSQKERLLDELTLEGVARYMQSERCRRVICLVGAGISTSAGIPDFRSPS
TGLYDNLEKYHLPYPEAIFEISYFKKHPEPFFALAKELYPGQFKPTICHY
FMRLLKDKGLLLRCYTQNIDTLERIAGLEQEDLVEAHGTFYTSHCVSASC
RHEYPLSWMKEKIFSEVTPKCEDCQSLVKPDIVFFGESLPARFFSCMQSD
FLJKVDLLLVMGTSLQVQPFASLISKAPLSTPRLLINKEKAGQSDPFLGM
IMGLGGGMDFDSKKAYRDVAWLGECDQGCLALAELLGWKKELEDLVRREH
ASIDAQSGAGVPNPSTSASPKKSPPPAKDEARTTEREKPQ
>sp|Q9NTG7|SIR3_HUMAN NAD-dependent deacetylase
sirtuin 3, mitochondrial precursor (EC 3.5.1.-)
(SIR2-like protein 3) (hSIRT3) - Homo sapiens
(Human).

(SEQ ID NO: 3)

MAFWGWRAAAALRLWGRVVERVEAGGGVGPFQACGCRLVLGGRDDVSAGL
RGSHGARGEPLDPARPLQRPPRPEVPRAFRRQPRAAAPSFFFSSIKGGRR
SISFSVGASSVVGSGGSSDKGKLSLQDVAELIRARACQRVVVMVGAGIST
PSGIPDFRSPGSGLYSNLQQYDLPYPEAIFELPFFFHNPKPFFTLAKELY
PGNYKPNVTHYFLRLLHDKGLLLRLYTQNIDGLERVSGIPASKLVEAHGT
FASATCTVCQRPFPGEDIRADVMADRVPRCPVCTGVVKPDIVFFGEPLPQ
RFLLHVVDFPMADLLLILGTSLEVEPFASLTEAVRSSVPRLLINRDLVGP
LAWHPRSRDVAQLGDVVHGVESLVELLGWTEEMRDLVQRETGKLDGPDK
>sp|Q9Y6E7|SIR4_HUMAN NAD-dependent deacetylase
sirtuin 4 (EC 3.5.1.-) (SIR2-like protein 4) -
Homo sapiens (Human).

(SEQ ID NO:4)

MKMSFALTFRSAKGRWIANPSQPCSKASIGLFVPASPPLDPEKVKELQRF
ITLSKRLLVMTGAGISTESGIPDYRSEKVGLYARTDRRPIQHGDFVRSAP
IRQRYWARNFVGWPQFSSHQPNPAHWALSTWEKLGKLYWLVTQNVDALHT
KAGSRRLTELHGCMDRVLCLDCGEQTPRGVLQERFQVLNPTWSAEAHGLA
PDGDVFLSEEQVRSFQVPTCVQCGGHLKPDVVFFGDTVNPDKVDFVHKRV
KEADSLLVVGSSLQVYSGYRFILTAWEKKLPIAILNIGPTRSDDLACLKL
NSRCGELLPLIDPC
>sp|Q9NXA8|SIR5_HUMAN NAD-dependent deacetylase
sirtuin 5 (EC 3.5.1.-) (SIR2-like protein 5) -
Homo sapiens (Human).

(SEQ ID NO:5)

MRPLQIVPSRLISQLYCGLKPPASTRNQICLKMARPSSSMADFRKFFAKA
KHIVIISGAGVSAESGVPTFRGAGGYWRKWQAQDATPLAFAHNPSRVWEF
YHYRREVMGSKEPNAGHRAIAECETRLGKQGRRVVVITQNIDELHRKAGT
KNLLEIHGSLFKTRCTSCGVVAENYKSPICPALSGKGAPEPGTQDASIPV
EKLPRCEEAGCGGLLRPHVVWFGENLDPAILEEVDRELAHCDLCLVVGTS
SVVYPAAMFAPQVAARGVPVAEFNTETTPATNRFRFHFQGPCGTTLPEAL
ACHENETVS
>sp|Q8N6T7|SIR6_HUMAN NAD-dependent deacetylase
sirtuin 6 (EC 3.5.1.-) (SIR2-like protein 6) -
Homo sapiens (Human)

(SEQ ID NO: 6)

MSVNYAAGLSPYADKGKCGLPEIFDPPEELERKVWELARLVWQSSSVVFH
TGAGISTASGIPDFRGPHGVWTMEERGLAPKFDTTFESARPTQTHMALVQ
LERVGLLRFLVSQNVDGLHVRSGFPRDKLAELHGNMFVEECAKCKTQYVR
DTVVGTMGLKATGRLCTVAKARGLRACRGELRDTILDWEDSLPDRDLALA
DEASRNADLSITLGTSLQIRPSGNLPLATKRRGGRLVIVNLQPTKHDRHA
DLRIHGYVDEVMTRLMKHLGLEIPAWDGPRVLERALPPLPRPPTPKLEPK
EESPTRINGSIPAGPKQEPCAQHNGSEPASPKRERPTSPAPHRPPKRVKA
KAVPS
>sp|Q9NRC8|SIR7 HUMAN NAD-dependent deacetylase
sirtuin 7 (EC 3.5.1.-) (SIR2-like protein 7) -
Homo sapiens (Human)

(SEQ ID NO: 7)

MAAGGLSRSERKAAERVRRLREEQQRERLRQVSRILRKAAAERSAEEGRL
LAESADLVTELQGRSRRREGLKRRQEEVCDDPEELRGKVRELASAVRNAK
YLVVYTGAGISTAASIPDYRGPNGVWTLLQKGRSVSAADLSEAEPTLTHM
SITRLHEQKLVQHVVSQNCDGLHLRSGLPRTAISELHGNMYIEVCTSCVP
NREYVRVFDVTERTALHRHQTGRTCHKCGTQLRDTIVHFGERGTLGQPLN
WEAATEAASRADTILCLGSSLKVLKKYPRLWCMTKPPSRRPKLYIVNLQW
TPKDDWAALKLHGKCDDVMRLLMAELGLEIPAYSRWQDPIFSLATPLRAG
EEGSHSRKSLCRSREEAPPGDRGAPLSSAPILGGWFGRGCTKRTKRKKVT

Exemplary compounds described herein may inhibit activity of SIRT1 or a functional domain thereof by at least 10, 20, 25, 30, 50, 80, or 90%, with respect to a natural or artificial substrate described herein. For example, the compounds may have a Ki of less than 500, 200, 100, or 50 nM.

A compound described herein may also modulate a complex between a sirtuin and a transcription factor, e.g., increase or decrease complex formation, deformation, and/or stability. Exemplary sirtuin-TF complexes include Sir2-PCAF, SIR2-MyoD, Sir2-PCAF-MyoD, and Sir2-p53. A compound described herein may also modulate expression of a Sir2 regulated gene, e.g., a gene described in Table 1 of Fulco et al. (2003) Mol. Cell 12:51-62.

In Vitro Assays

In some embodiments, interaction with, e.g., binding of, SIRT1 can be assayed in vitro. The reaction mixture can include a SIRT1 co-factor such as NAD and/or a NAD analog.

In other embodiments, the reaction mixture can include a SIRT1 binding partner, e.g., a transcription factor, e.g., a viral transcription factor (e.g., tat), p53 or a transcription factor other than p53, and compounds can be screened, e.g., in an in vitro assay, to evaluate the ability of a test compound to modulate interaction between SIRT1 and a SIRT1 binding partner, e.g., a transcription factor. This type of assay can be accomplished, for example, by coupling one of the components, with a radioisotope or enzymatic label such that binding of the labeled component to the other can be determined by detecting the labeled compound in a complex. A component can be labeled with ¹²⁵I, ³⁵S, ¹⁴C, or ³H, either directly or indirectly, and the radioisotope detected by direct counting of radioemmission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

Cell-free assays involve preparing a reaction mixture of the target protein (e.g., SIRT1) and the test compound under conditions and for a time sufficient to allow the two components to interact and bind, thus forming a complex that can be removed and/or detected.

The interaction between two molecules can also be detected, e.g., using a fluorescence assay in which at least one molecule is fluorescently labeled. One example of such an assay includes fluorescence energy transfer (FET or FRET for fluorescence resonance energy transfer) (see, for example, Lakowicz et al., U.S. Pat. No. 5,631,169; Stavrianopoulos, et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FET binding event can be conveniently measured through standard fluorometric detection means well known in the art (e.g., using a fluorimeter).

Another example of a fluorescence assay is fluorescence polarization (FP). For FP, only one component needs to be labeled. A binding interaction is detected by a change in molecular size of the labeled component. The size change alters the tumbling rate of the component in solution and is detected as a change in FP. See, e.g., Nasir et al. (1999) Comb Chem HTS 2:177-190; Jameson et al. (1995) Methods Enzymol 246:283; Seethala et al. (1998) Anal Biochem. 255:257. Fluorescence polarization can be monitored in multiwell plates, e.g., using the Tecan Polarion™ reader. See, e.g., Parker et al. (2000) Journal of Biomolecular Screening 5:77-88; and Shoeman, et al. (1999) 38, 16802-16809.

In another embodiment, determining the ability of the SIRT1 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, SIRT1 is anchored onto a solid phase. The SIRT1/test compound complexes anchored on the solid phase can be detected at the end of the reaction, e.g., the binding reaction. For example, SIRT1 can be anchored onto a solid surface, and the test compound, (which is not anchored), can be labeled, either directly or indirectly, with detectable labels discussed herein.

It may be desirable to immobilize either the SIRT1 or an anti-SIRT1 antibody to facilitate separation of complexed from uncomplexed forms of one or both of the proteins, as well as to accommodate automation of the assay. Binding of a test compound to a SIRT1 protein, or interaction of a SIRT1 protein with a second component in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-5-transferase/SIRT1 fusion proteins or glutathione-5-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or SIRT1 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of SIRT1 binding or activity determined using standard techniques.

Other techniques for immobilizing either a SIRT1 protein or a target molecule on matrices include using conjugation of biotin and streptavidin. Biotinylated SIRT1 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with a SIRT1 protein or target molecules but which do not interfere with binding of the SIRT1 protein to its target molecule. Such antibodies can be derivatized to the wells of the plate, and unbound target or the SIRT1 protein trapped in the wells by antibody conjugation. Methods for detecting such complexes, in addition to those described above for the GST-immobilized complexes, include immunodetection of complexes using antibodies reactive with the SIRT1 protein or target molecule, as well as enzyme-linked assays which rely on detecting an enzymatic activity associated with the SIRT1 protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

In a preferred embodiment, the assay includes contacting the SIRT1 protein or biologically active portion thereof with a known compound which binds a SIRT1 to form an assay mixture, contacting the assay mixture with a test compound, and determining the ability of the test compound to interact with a SIRT1 protein, wherein determining the ability of the test compound to interact with the SIRT1 protein includes determining the ability of the test compound to preferentially bind to the SIRT1 or biologically active portion thereof, or to modulate the activity of a target molecule, as compared to the known compound.

An exemplary assay method includes a 1536 well format of the SirT1 enzymatic assay that is based on the commercial “Fluor-de-Lys” assay principle by Biomol, which is fluorogenic (www.biomol.com/store/Product_Data_PDFs/ak500.pdf). In this assay, deacetylation of the e-amino function of a lysyl residue is coupled to a fluorogenic “development step that is dependent on the unblocked e-amino functionality and generates fluorescent aminomethylcoumarin. Fluorescence can be read on a commercial macroscopic reader.

Additional Assays

A compound or library of compounds described herein can also be evaluated using model systems for a disease or disorder, or other known models of a disease or disorder described herein.

Structure-Activity Relationships and Structure-Based Design. It is also possible to use structure-activity relationships (SAR) and structure-based design principles to produce a compound that interact with a sirtuin, e.g., antagonizes or agonizes a sirtuin. SARs provide information about the activity of related compounds in at least one relevant assay. Correlations are made between structural features of a compound of interest and an activity. For example, it may be possible by evaluating SARs for a family of compounds related to a compound described herein to identify one or more structural features required for the agonist's activity. A library of compounds can then be chemically produced that vary these features. In another example, a single compound that is predicted to interact is produced and evaluated in vitro or in vivo.

Structure-based design can include determining a structural model of the physical interaction of a functional domain of a sirtuin and a compound. The structural model can indicate how the compound can be engineered, e.g., to improve interaction or reduce unfavorable interactions. The compound's interaction with the sirtuin can be identified, e.g., by solution of a crystal structure, NMR, or computer-based modeling, e.g., docking methods. See, e.g., Ewing et al. J Comput Aided Mol Des. 2001 May; 15(5):411-28.

Both the SAR and the structure-based design approach, as well as other methods, can be used to identify a pharmacophore. A pharmacophore is defined as a distinct three dimensional (3D) arrangement of chemical groups. The selection of such groups may be favorable for biological activity. Since a pharmaceutically active molecule must interact with one or more molecular structures within the body of the subject in order to be effective, and the desired functional properties of the molecule are derived from these interactions, each active compound must contain a distinct arrangement of chemical groups which enable this interaction to occur. The chemical groups, commonly termed descriptor centers, can be represented by (a) an atom or group of atoms; (b) pseudo-atoms, for example a center of a ring, or the center of mass of a molecule; (c) vectors, for example atomic pairs, electron lone pair directions, or the normal to a plane. Once formulated a pharmacophore can be used to search a database of chemical compound, e.g., for those having a structure compatible with the pharmacophore. See, for example, U.S. Pat. No. 6,343,257; Y C. Martin, 3D Database Searching in Drug Design, J. Med. Chem. 35, 2145(1992); and A. C. Good and J. S. Mason, Three Dimensional Structure Database Searches, Reviews in Comp. Chem. 7, 67(1996). Database search queries are based not only on chemical property information but also on precise geometric information.

Computer-based approaches can use database searching to find matching templates; Y. C. Martin, Database searching in drug design, J. Medicinal Chemistry, vol. 35, pp 2145-54 (1992), which is herein incorporated by reference. Existing methods for searching 2-D and 3-D databases of compounds are applicable. Lederle of American Cyanamid (Pearl River, N.Y.) has pioneered molecular shape-searching, 3D searching and trend-vectors of databases. Commercial vendors and other research groups also provide searching capabilities (MACSS-3D, Molecular Design Ltd. (San Leandro, Calif.); CAVEAT, Lauri, G. et al., University of California (Berkeley, Calif.); CHEM-X, Chemical Design, Inc. (Mahwah, N.J.)). Software for these searches can be used to analyze databases of potential drug compounds indexed by their significant chemical and geometric structure (e.g., the Standard Drugs File (Derwent Publications Ltd., London, England), the Bielstein database (Bielstein Information, Frankfurt, Germany or Chicago), and the Chemical Registry database (CAS, Columbus, Ohio)).

Once a compound is identified that matches the pharmocophore, it can be tested for activity in vitro, in vivo, or in silico, e.g., for binding to a sirtuin or domain thereof.

In one embodiment, a compound that is an agonist or a candidate agonist, e.g., a compound described in Nature. 2003 Sep. 11; 425(6954):191-196 can be modified to identify an antagonist, e.g., using the method described herein. For example, a library of related compounds can be prepared and the library can be screened in an assay described herein.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

The compounds can be administered alone, or in combination with on or more additional therapeutic agents, e.g., a protease inhibitor, e.g., a HIV protease inhibitor, a fusion inhibitor, an integrase inhibitor, or a reverse transcriptase inhibitor, (e.g., a nucleotide analog, e.g., AZT, or a non-nucleoside reverse transcriptase inhibitor). When a compound is administered in combination with another (e.g., at least one additional) therapeutic agent the compound and agent can be administered in a single composition, for example a single pill or suspension, or the compound and agent (or agents) can be administered separately, for example in multiple compositions such as pills or suspensions. When administered separately, the compound and agent (or agents) can be administered at the same time, or at different times. In some instances, the compound and agent (or agents) have the same course of therapy, and in other times, the courses are either skewed or sequential.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions delineated herein include the compounds of the formulae delineated herein, as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-(X-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Viral Disorders

The compounds of the invention can be used in the treatment of a viral disease or disorder For example, the disease or disorder can be a retroviral disorder, e.g., an HIV-mediated disorder such as AIDS because SIRT1 deacetylates the HIV Tat protein and is required for Tat-mediated Transactivation of the HIV Promoter. The compounds of the invention can also be used to treat a Tat-mediated or Tat-related disorder.

A compound described herein can be formulated with one or more other anti-viral agents. In another implementation the compound is administered in conjunction with (e.g., concurrently with) one or more anti-viral agents, e.g., as separate formulations. Exemplary anti-viral agents include drugs for treating AIDS such as:

| | | Also | |
|---|---|---|---|
| Generic Name | Trade Name | Known As: | Manufacturer |
| saquinavir | INVIRASE ® | SQV | Roche |
| ritonavir | NORVIR ® | RTV | Abbott |
| indinavir | CRIXIVAN ® | IDV | Merck |
| nelfinavir | VIRACEPT ® | NFV | Pfizer |
| saquinavir | FORTOVASE ® | SQV | Roche |
| amprenavir | AGENERASE ® | APV, | GlaxoSmithKline |
| | | 141W94 | |
| lopinavir | KALETRA ® | ABT-378/r | Abbott |
| tenofovir | VIREAD ® | | Gilead |
| disoproxil | | | |
| emtricitabine | EMTRIVA ® | Gilead | |
| a fixed dose of | TRUVADA ® | Gilead | |
| emtricitabine and | | | |
| tenofovir | | | |
| disoproxil | | | |
| fumarate | | | |

ATAZANAVIR® (BMS 232632) by Bristol-Myers Squibb, GW433908 by GlaxoSmithKline, L-756,423 by Merck, MOZENAVIR (DMP-450) by Triangle Pharmaceuticals, TIPRANAVIRS by Boehringer Ingelheim and TMC114 by Tibotec Virco.

The invention includes, inter alia, methods for modulating activity of a virus. For example, the compounds of the invention can be used to modulate the acetylation state of a viral factor. An exemplary viral factor that is a substrate for sirtuins is HIV tat

An exemplary amino acid sequence of HIV-1 tat is as follows:

(SEQ ID NO: 8)

MEPVDPNLEPWNHPGSQPTTACSNCYCKVCCWHCQLCFMTKGLSISYGRK
KRKRRRGTPHGSEDHQNLISKQPSSQPRGDPTGPKEQKKKVESKAEADPF
D

An exemplary amino acid sequence of HIV-2 tat is as follows:

(SEQ ID NO:9)

MGIPLQEQENSLEFSSERSSSTSEEGANTRGLDNQGEEILSQLYRPLEAC
RNKCYCKKCCYHCQLCFLKKGLGICYDHSRKRSSKRAKVTAPTASNDLST
RARDGQPAKKQKKEVETTRTTDPGLGRSDTSTS.

Kits

A compound described herein described herein can be provided in a kit. The kit includes (a) a compound described herein, e.g., a composition that includes a compound described herein, and, optionally (b) informational material. The informational material can be descriptive, instructional, marketing or other material that relates to the methods described herein and/or the use of a compound described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound described herein in a suitable manner to perform the methods described herein, e.g., in a suitable dose, dosage form, or mode of administration (e.g., a dose, dosage form, or mode of administration described herein). In another embodiment, the informational material can include instructions to administer a compound described herein to a suitable subject, e.g., a human, e.g., a human having or at risk for a disorder described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound described herein, the composition of the kit can include other ingredients, such as a solvent or buffer, a stabilizer, a preservative, a flavoring agent (e.g., a bitter antagonist or a sweetener), a fragrance or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

A compound described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

The fact that a patient has been treated with a molecule of the invention, or the patient's response to treatment with a molecule of the invention, can be used, alone or in combination with other information, e.g., other information about the patient, to determine whether to authorize or transfer of funds to pay for a service or treatment provided to a subject. For example, an entity, e.g., a hospital, care giver, government entity, or an insurance company or other entity which pays for, or reimburses medical expenses, can use such information to determine whether a party, e.g., a party other than the subject patient, will pay for services or treatment provided to the patient. For example, a first entity, e.g., an insurance company, can use such information to determine whether to provide financial payment to, or on behalf of, a patient, e.g., whether to reimburse a third party, e.g., a vendor of goods or services, a hospital, physician, or other caregiver, for a service or treatment provided to a patient. For example, a first entity, e.g., an insurance company, can use such information to determine whether to authorize, recommend, pay, reimburse, continue, discontinue, enroll an individual in an insurance plan or program, e.g., a health insurance or life insurance plan or program.

Databases

The invention also features a database that associates information about or identifying one or more of the compounds described herein with a parameter about a patient, e.g., a patient being treated with a disorder herein. The parameter can be a general parameter, e.g., blood pressure, core body temperature, etc., or a parameter related to a viral disease or disorder, e.g., as described herein, e.g., e.g., viral load or white blood cell count.

All references cited herein, whether in print, electronic, computer readable storage media or other form, are expressly incorporated by reference in their entirety, including but not limited to, abstracts, articles, journals, publications, texts, treatises, internet web sites, databases, patents, patent applications, and patent publications.

### EXAMPLES

### Example 1

List of Reagents:

| | | Supplied | | Catalog | |
|---|---|---|---|---|---|
| | Name of Reagent | As | Source | Number | Storage |
| 1 | human SirT1 | 2.5 or 3.5 U/ul | Biomol | SE-239 | −20 C. |
| 2 | Fluor de Lys | 50 mM in | Biomol | KI-104 | −20 C. |
| | Substrate | DMSO | | | |
| 3 | Fluor de Lys | 20×Biomol | KI-105 | −20 C. | |
| | Developer | concentrate | | | |
| 4 | NAD | solid | Sigma | N-1636 | −20 C. |
| 5 | Nicotinamide | solid | Calbiochem | 481907 | RT |
| 6 | Trizma-HCI | solid | Sigma | T-5941 | RT |
| 7 | Sodium | solid | Sigma | S-9888 | RT |
| | Chloride | | | | |
| 8 | Magnesium | solid | Sigma | M-2393 | RT |
| | Chloride | | | | |
| 9 | Potassium | solid | Sigma | P-3911 | RT |
| | Chloride | | | | |
| 10 | Polyoxyethylene | 100% | Sigma | P-7949 | RT |
| | sorbitan | | | | |
| | monolaurate | | | | |
| | (Tween-20) | | | | |
| 11 | Fluor de Lys | 10 mM in | Biomol | KI-142 | −20 C. |
| | Deacetylated | DMSO | | | |
| | Standard | | | | |

List of Equipment:

| | | | Catalog |
|---|---|---|---|
| | Tool Name | Tool Source | Number |
| 1 | Fluorescence Plate Reader | BIO-TEK | SIAFR |
| | Synergy HT | | |
| 2 | Matrix Impact2 16 Channel | Apogent Discoveries | 2069 |
| | pipet | | |
| 3 | 37° C. Incubator | VWR | 1540 |

List of Disposables:

| | Disposable | Source | Catalog Number |
|---|---|---|---|
| 1 | 384 white low volume | Greiner/Bellco | 4507-84075 |
| | plates | | |
| 2 | Tips for matrix 16 chan | Apogent Discoveries | 7421 |
| | pipet | | |
| 3 | 25 ml divided reagent | Apogent Discoveries | 8095 |
| | reservoirs | | |
| 4 | Plate Sealing Films | Apogent Discoveries | 4418 |

Standard Reagent Formulations:

| | | | | | Final | |
|---|---|---|---|---|---|---|
| | | | | Compo- | Compo- | |
| | | | | nent | nent | |
| | Prepared | Component | | Quantity | Concen- | Stor- |
| | Reagent Name | Name | M.W. | (in water) | tration | age |
| 1 | Tris-HCl, | Trizma-HCl | 157.6 | 157.6 g/L | 1 M | RT |
| | pH 8.0 | HCl | | to pH 8.0 | pH 8.0 | |
| 2 | Sodium | NaCl | 58.44 | 292 g/L | 5 M | RT |
| | Chloride | | | | | |
| 3 | Magnesium | MgCl₂ | 203.3 | 20.33 g/L | 100 | RT |
| | Chloride | | | | mM | |
| 4 | Potassium | KCl | 74.55 | 20.13 g/L | 270 | RT |
| | Chloride | | | | mM | |
| 5 | Polyoxyethyl- | Tween-20 | | 1 ml/ | 10% | RT |
| | ene sorbitan | | | 10 ml | | |
| | monolaurate | | | | | |
| 6 | NAD | NAD | 717 | 0.0717 | 100 | −20 |
| | | | g/ml | mM | C. | |
| 7 | Nicotinamide | Nicotina- | 122 | 0.0061 | 50 mM | −20 |
| | | mide | | g/ml | | C. |
| 8 | Assay Buffer | Tris-HCl, pH | | 25 ml of 1 | 25 mM | 4 C. |
| | | 8.0 | | M stock/L | | |
| | | NaCl | | 27.4 ml of | 137 | |
| | | | | 5 M | mM | |
| | | | | stock/L | | |
| | | KCl | | 10 ml of | 2.7 mM | |
| | | | | 270 mM | | |
| | | | | stock/L | | |
| | | MgCl₂ | | 10 ml of | 1 mM | |
| | | | | 100 mM | | |
| | | | | stock/L | | |
| | | Tween-20 | | 5 ml of | 0.05% | |
| | | | | 10% | | |
| | | | | stock/L | | |

| **Prepare working stocks below | | | The fol- | | |
|---|---|---|---|---|---|
| just before use | | | lowing are | | |
| | | | prepared | | |
| | | | in assay | | |
| | | | buffer | | |
| 9 | 2× Substrates | Flour de Lys | 6 ul/ml | 300 uM | ice |
| | | substrate | | | |
| | | NAD | 20 ul of | 2 mM | |
| | | | 100 mM | | |
| | | | stock/ml | | |
| 10 | Enzyme Mix | Biomol | **depends | 0.125 | ice |
| | | SirT1 | upon spe- | (0.5 U/ | U/ul |
| | | | cific activ- | well) | |
| | | | ity of lot. | | |
| | | | Ex: 3.5 U/ | | |
| | | | ul, 35.7 | | |
| | | | ul/ml | | |
| 11 | Developer/ | 20× de- | 50 ul/ml | 1× in | ice |
| | stop reagent | veloper | assay | | |
| | | concentrate | buffer | | |
| | | nicotinamide | 20 ul of | 1 mM | |
| | | | 50 mM | | |
| | | | stock/ml | | |

Procedure Description:

Step Description
1 Prepare amount of 2× Substrates necessary for the number of wells to be assayed. 5 ul per well is needed
2 Dispense 5 ul 2× substrates to test wells
3 Dispense 1 ul of test compound to the test wells
   Dispense 1 ul of compound solvent/diluent to the positive control wells
   Dispense 1 ul of 1 mM nicotinamide to the 50% inhibition wells
   Dispense 1 ul of 10 mM nicotinamide to the 100% inhibition wells
4 Dispense 4 ul of assay buffer to negative control wells (no enzyme controls)
5 Prepare amount of enzyme necessary for number of wells to assay. 4 ul enzyme mix needed per well
6 Dispense 4 ul of enzyme mix to the test wells and positive control wells
7 Cover and incubate at 37C for 45 minutes
8 Less then 30 minutes before use, prepare amount of 1× developer/stop reagent for the number of wells being assayed
9 Dispense 10 ul 1× developer/stop reagent to all wells
10 Incubate at room temperature for at least 15 minutes
11 Read in fluorescence plate reader, excitation=350-380 nm, emission=440-460
12 Fluor de Lys in the substrate has an intrinsic fluorescence that needs to be subtracted as background before any calculations are to be done on the data. These values can be found in the negative control wells.

Appendix 1: Preparation of a Standard Curve Using Fluor de Lys Deactylated Standard
1 Determine the concentration range of deactylated standard to use in conjunction with the above assay by making a 1 uM dilution of the standard. Mix 10 ul of the 1 uM dilution with 10 ul developer and read at the same wavelengths and sensitivity settings that the assay is read at. Use this estimate of AFU (arbitrary fluorescence units)/uM to determine the range of concentrations to test in the standard curve.
2 Prepare, in assay buffer, a series of dilutions of the Fluor de Lys deactylated standard that span the desired concentration range
3 Pipet 10 ul assay buffer to the ‘zero’ wells.
4 Pipet 10 ul of the standard dilutions into wells
5 Pipet 10 ul developer to the wells and incubate 15 minutes at RT
6 Read plate at above wavelengths
7 Plot fluorescence signal (y) versus concentration of the Fluor de Lys deacetylated standard (x) and determine the slope as AFU/uM

Protocol for Testing for Inhibitors of the Developer Reaction
1 From the standard curve select concentration of deacetylated standard that gives a fluorescence signal equivalent to positive controls in assay (eg. 5 uM)
2 Dispense 5 ul 2× deacetylated standard (eg. 10 uM)
3 Dispense 1 ul compound, 4 ul assay buffer
4 Dispense 10 ul developer
5 Incubate at room temp 15 minutes (or equivalent time as in screen) and read at same settings as screen

### Example 2

HeLa cells were transfected with GFP-hSIRT2 isoform 1. At 36 hours post transfection 1 μM of TSA and either DMSO or 50 μM of Compound 8 was added. The next morning cells were fixed, permeabilized, and stained for acetylated tubulin. In cells treated with DMSO there was very little acetylated tubulin in cells expressing SIRT2, in cells treated with Compound 8 the tubulin is more highly acetylated indicating that the effect of SIRT2 was blocked. See FIG. 2.

It was also possible to observe the effect of the compounds using Western analysis. 293T cells were transfected with either eGFP (control) or with mouse SIRT2 Isoform 1 (mSIRT2). TSA was added to increase amount of acetylated tubulin and at the same time either DMSO or the compound listed below were added to 10 μM.

What is claimed is:

1. A method for treating an HIV-mediated disorder in a subject, the method comprising administering to the subject an effective amount of a compound having a formula (I): 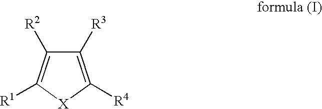
wherein;
R¹ is H, halo, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl; or when taken together with R² and the carbon to which it is attached, forms C₅-C₁₀ cycloalkenyl, C₅-C₁₀ heterocycloalkenyl, C₆-C₁₀ aryl, or C₆-C₁₀ heteroaryl; each of which can be optionally substituted with 1-5 R⁵;
R² is H, halo, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl; or when taken together with R² and the carbon to which it is attached, forms C₅-C₁₀ cycloalkenyl, C₅-C₁₀ heterocycloalkenyl, C₆-C₁₀ aryl, or C₆-C₁₀ heteroaryl; each of which can be optionally substituted with 1-5 R⁶;
each of R³ and R⁴ is, independently, H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃R⁹, sulfate, S(O)N(R⁹)₂, S(O)₂N(R⁹)₂, phosphate, C₁-C₄ alkylenedioxy, acyl, amido, aminocarbonyl, C₁-C₆ alkyl aminocarbonyl, C₁-C₆ dialkyl aminocarbonyl, aminocarbonylalkyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl; each of which is independently substituted with one or more R⁷;
each or R⁵ and R⁶ is, independently, halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, oxo, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃R⁹, sulfate, S(O)N(R⁹)₂, S(O)₂N(R⁹)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl;
each R⁷ is independently C₁-C₁₀ alkyl, C₁-C₆ haloalkyl, aminocarbonyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₇-C₁₂ heterocyclylalkyl, C₇-C₁₂ cyloalkylalkyl, C₇-C₁₂ heterocycloalkenylalkyl, or C₇-C₁₂ cycloalkenylalkyl; each of which is optionally substituted with 1-4 R¹⁰;
X is NR⁸, O, or S;
R⁸ is H, C₁-C₆ alkyl, C₆-C₁₀ aryl, C₅-C₁₀ heteroaryl, C₇-C₁₂ arylalkyl, C₇-C₁₂ heteroarylalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₇-C₁₂ heterocyclylalkyl, C₇-C₁₂ cyloalkylalkyl, C₇-C₁₂ heterocycloalkenylalkyl, or C₇-C₁₂ cycloalkenylalkyl;
R⁹ is H or C₁-C₆ alkyl; and
each R¹⁰ is independently halo, hydroxy, alkoxy, alkyl, alkenyl, alkynl, nitro, amino, cyano, amido, or aminocarbonyl.

2. The method of claim 1, wherein R¹ and R², taken together, with the carbons to which they are attached, form C₅-C₁₀ cycloalkenyl, C₅-C₁₀ heterocycloalkenyl, C₆-C₁₀ aryl, or C₆-C₁₀ heteroaryl.

3. The method of claim 2, wherein R¹ and R², taken together, with the carbons to which they are attached, form C₅-C₁₀ cycloalkenyl.

4. The method of claim 3, wherein R¹ and R², taken together, with the carbons to which they are attached, form C₅-C₁₀ cycloalkenyl, optionally substituted with 1 or 2 C₁-C₆ alkyl.

5. The method of claim 4, wherein R¹ and R², taken together form a C₅-C₇ cycloalkenyl ring substituted with C₁-C₆ alkyl.

6. The method of claim 1, wherein R¹ is C₆-C₁₀ aryl, C₅-C₁₀ heteroaryl, C₇-C₁₂ aralkyl, C₇-C₁₂ heteroaralkyl, C₃-C₈ heterocyclyl, C₅-C₁₀ cycloalkenyl, or C₅-C₁₀ heterocycloalkenyl.

7. The method of claim 6, wherein R¹ is C₆-C₁₀ aryl.

8. The method of claim 1, wherein R² is H, halo, C₁-C₁₀ alkyl, or C₁-C₆ haloalkyl.

9. The method of claim 1, wherein R³ is carboxy, cyano, aminocarbonyl, C₁-C₆ alkyl aminocarbonyl, C₁-C₆ dialkyl aminocarbonyl, C₁-C₁₀ alkoxycarbonyl, C₁-C₁₀ alkylthioylcarbonyl, hydrazinocarbonyl, C₁-C₆ alkylhydrazinocarbonyl, C₁-C₆ dialkyl hydrazinocarbonyl, or hydroxyaminocarbonyl.

10. The method of claim 9, wherein R³ is aminocarbonyl, C₁-C₆ alkyl aminocarbonyl, C₁-C₆ dialkyl aminocarbonyl, hydrazinocarbonyl, C₁-C₆ alkyl hydrazinocarbonyl, C₁-C₆ dialkyl hydrazinocarbonyl, or hydroxyaminocarbonyl.

11. The method of claim 10, wherein R³ is aminocarbonyl, C₁-C₆ alkyl aminocarbonyl, or C₁-C₆ dialkyl aminocarbonyl.

12. The method of claim 1, wherin R³ is H, thioalkoxy or thioaryloxy.

13. The method of claim 1, wherein R⁴ is nitro, amino, C₁-C₆ alkyl amino, C₁-C₆ dialkyl amino, or amido.

14. The method of claim 13, wherein R⁴ is amino or amido.

15. The method of claim 1, wherein R⁴ is aminocarbonylalkyl.

16. The method of claim 15, wherein amino of the aminocarbonylalkyl is substituted with aryl, arylalkyl, alkyl, etc.

17. The method of claim 16, wherein each substituent can independently be further substituted with halo, hydroxy, or alkoxy.

18. The method of claim 1, wherein
R³ is aminocarbonyl, C₁-C₆ alkyl aminocarbonyl, or C₁-C₆ dialkyl aminocarbonyl; and
R⁴ is amino, C₁-C₆ alkyl amino C₁-C₆ dialkyl amino or amido.

19. The method of claim 1, wherein X is S.

20. The method of claim 1, wherein X is NR⁸.

21. The method of claim 20, wherein R⁸ is H, C₁-C₆ alkyl or C₇-C₁₀ arylalkyl.

22. The method of claim 1, wherein
R¹ is C₆-C₁₀ aryl, C₅-C₁₀ heteroaryl, C₇-C₁₂ aralkyl, C₇-C₁₂ heteroaralkyl, C₃-C₈ heterocyclyl, C₅-C₁₀ cycloalkenyl, or C₅-C₁₀ heterocycloalkenyl; or when taken together with R² and the carbon to which it is attached, forms C₅-C₁₀ cycloalkenyl;
R² is H, halo, C₁-C₁₀ alkyl, C₁-C₆ haloalkyl; or when taken together with R¹ and the carbon to which it is attached, forms C₅-C₁₀ cycloalkenyl;
R³ is aminocarbonyl, C₁-C₆ alkyl aminocarbonyl, C₁-C₆ dialkyl aminocarbonyl, hydrazinocarbonyl, C₁-C₆ alkyl hydrazinocarbonyl, C₁-C₆ dialkyl hydrazinocarbonyl, or hydroxyaminocarbonyl;
R⁴ is amino, C₁-C₆ alkyl amino, C₁-C₆ dialkyl amino, or amido; and
X is S.

23. The method of claim 1, wherein
R¹ and R², taken together with the carbons to which they are attached, form C₅-C₁₀ cycloalkenyl;
R³ is aminocarbonyl, C₁-C₆ alkyl aminocarbonyl, or C₁-C₆ dialkyl aminocarbonyl;
R⁴ is amino, C₁-C₆ alkyl amino, C₁-C₆ dialkyl amino, or amido; and
X is S.

24. The compound of claim 1, wherein the compound preferentially inhibits SirT1 relative to a non-SirT1 sirtuin.

25. The compound of claim 1, wherein the compound has at least a 5 fold preference for SirT1.

26. The compound of claim 1, wherein the compound has a Kᵢ for SirT1 of less than about 1 μM.

27. A method for treating an HIV-mediated disorder in a subject, the method comprising administering to the subject an effective amount of a compound having a formula (II): 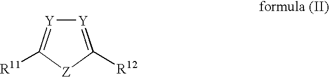
wherein;
R¹¹ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃(R¹³), sulfate, S(O)N(R¹³)₂, S(O)₂N(R¹³)₂, phosphate, C₁-C₄ alkylenedioxy, acyl, amido, aminocarbonyl, aminocarbonylalkyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl; wherein each is optionally substituted with R¹⁴;
R¹² is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃(R³), sulfate, S(O)N(R³)₂, S(O)₂N(R³)₂, phosphate, C₁-C₄ alkylenedioxy, acyl, amido, aminocarbonyl, aminocarbonylalkyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, or hydroxyaminocarbonyl or alkoxyaminocarbonyl; wherein each is optionally substituted with R¹⁵;
R¹³ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl;
R¹⁴ is hydroxy, carboxy, carboxylate, cyano, nitro, amino, C₁-C₆ alkyl amino, C₁-C₆ dialkyl amino, oxo, mercapto, thioalkoxy, thioaryloxy, thioheteroaryloxy, SO₃H, sulfate, S(O)NH₂, S(O)₂NH₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl;
R¹⁵ is halo, hydroxy, C₁-C₁₀ alkyl, C₁-C₆ haloalkyl, C₁-C₁₀ alkoxy, C₁-C₆ haloalkoxy, C₆-C₁₀ aryloxy, C₅-C₁₀ heteroaryloxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ arylalkoxy, or C₅-C₁₀ heteroarylalkoxy;
Z is NR¹⁶, O, or S;
each Y is independently N or CR¹⁸;
R¹⁶ is H, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₅-C₁₀ cycloalkenyl, C₅-C₁₀ heterocycloalkenyl, C₂-C₁₂ alkenyl, C₂-C₁₂ alkynyl; or one of R¹¹ or R¹² and R¹⁶ form a cyclic moiety containing 4-6 carbons, 1-3 nitrogens, 0-2 oxygens and 0-2 sulfurs; wherein each is optionally substituted with R¹⁷;
R¹⁷ is halo, hydroxy, C₁-C₆ alkyl, C₁-C₆ haloalkyl, C₁-C₆ alkoxy, C₁-C₆ haloalkoxy, C₂-C₈ alkenyl, C₂-C₈ alkynyl, oxo, mercapto, thioalkoxy, SO₃H, sulfate, S(O)NH₂, S(O)₂NH₂, phosphate, acyl, amido, aminocarbonyl, C₁-C₆ alkyl aminocarbonyl, C₁-C₆ dialkyl aminocarbonyl, C₁-C₆ alkoxycarbonyl, C₁-C₆ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl; and
R¹⁸ is H, halo, or C₁-C₆ alkyl.

28. The method of claim 27, wherein Z is NR¹⁶.

29. The method of claim 28, wherein Z is NR¹⁶, and R¹⁶ is C₁-C₁₀ alkyl, cycloalkenyl, C₅-C₁₀ heterocycloalkenyl, C₆-C₁₀ aryl, C₅-C₁₀ heteroaryl, C₇-C₁₂ aralkyl, or C₇-C₁₂ heteroaralkyl.

30. The method of claim 29, wherein R¹⁶ is C₁-C₁₀ alkyl, C₆-C₁₀ aryl, C₅-C₁₀ heteroaryl, C₇-C₁₂ aralkyl, or C₇-C₁₂ heteroaralkyl, substituted with one or more halo, alkyl, or alkoxy.

31. The method of claim 27, wherein R¹¹ is mercapto, thioalkoxy, thioaryloxy, thioheteroaryloxy, SO₃(R¹³), sulfate, S(O)N(R¹³)₂, S(O)₂N(R¹³)₂.

32. The method of claim 31, wherein R¹¹ is thioalkoxy, thioaryloxy, thioheteroaryloxy.

33. The method of claim 32, wherein R¹¹ is thioalkoxy, thioaryloxy, thioheteroaryloxy; substituted with one or more acyl, amido aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl.

34. The method of claim 33, wherein R¹¹ is thioalkoxy substituted with one or more amido, aminocarbonyl, C₁-C₆ alkyl aminocarbonyl, or C₁-C₆ dialkyl aminocarbonyl.

35. The method of claim 34, wherein R¹¹ is thioalkoxy substituted with aminocarbonyl.

36. The method of claim 27, wherein R¹² is C₁-C₁₀ alkyl, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl.

37. The method of claim 36, wherein R¹² is C₁-C₁₀ alkyl, C₆-C₁₀ aryl, C₅-C₁₀ heteroaryl, C₇-C₁₂ aralkyl, or C₇-C₁₂ heteroaralkyl.

38. The method of claim 37, wherein R¹² is C₁-C₁₀ alkyl substituted with one or more halo, hydroxy, C₁-C₁₀ alkyl, C₁-C₆ haloalkyl, C₁-C₁₀ alkoxy, C₆-C₁₀ aryloxy, or C₅-C₁₀ heteroaryloxy.

39. The method of claim 38, wherein R¹² is C₁-C₁₀ alkyl substituted with aryloxy.

40. The method of claim 27, wherein each Y is N.

41. The method of claim 27, wherein
R¹¹ is thioalkoxy, thioaryloxy, thioheteroaryloxy; substituted with one or more acyl, amido aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl;
R¹² is C₁-C₁₀ alkyl substituted with one or more halo, hydroxy, C₁-C₁₀ alkyl, C₁-C₆ haloalkyl, C₁-C₁₀ alkoxy, C₆-C₁₀ aryloxy, or C₅-C₁₀ heteroaryloxy
Z is NR¹⁶;
each Y is N; and
R¹⁶ is C₁-C₁₀ alkyl, C₆-C₁₀ aryl, C₅-C₁₀ heteroaryl, C₇-C₁₂ aralkyl, or C₇-C₁₂ heteroaralkyl, substituted with one or more halo, alkyl, or alkoxy.

42. A method for treating an HIV-mediated disorder in a subject, the method comprising administering to the subject an effective amount of a compound having a formula (III): 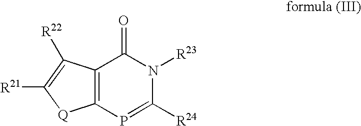
wherein;
R²¹ is halo, C₁-C₁₀ alkyl, C₁-C₆ haloalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryl, C₅-C₁₀ heteroaryl, C₇-C₁₂ aralkyl, C₇-C₁₂ heteroaralkyl; or when taken together with R²² and the carbon to which it is attached, forms C₅-C₁₀ cycloalkenyl, C₅-C₁₀ heterocycloalkenyl, C₆-C₁₀ aryl, or C₅-C₁₀ heteroaryl; each of which can be optionally substituted with 1-5 R²⁵;
R²² is halo, C₁-C₁₀ alkyl, C₁-C₆ haloalkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryl, C₅-C₁₀ heteroaryl, C₇-C₁₂ aralkyl, C₇-C₁₂ heteroaralkyl; or when taken together with R²¹ and the carbon to which it is attached, forms C₅-C₁₀ cycloalkenyl, C₅-C₁₀ heterocycloalkenyl, C₆-C₁₀ aryl, or C₅-C₁₀ heteroaryl; each of which is optionally substituted with 1-5 R²⁶;
R²³ is H, halo, hydroxy, C₁-C₁₀ alkyl, C₁-C₆ haloalkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, amino, C₁-C₆ alkyl amino, C₁-C₆ dialkyl amino, acyl, C₁-C₁₀ alkoxycarbonyl, C₁-C₁₀ thioalkoxycarbonyl;
R²⁴ is, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, amino, C₁-C₆ alkyl amino, C₁-C₆ dialkyl amino, mercapto, thioalkoxy, thioaryloxy, thioheteroaryloxy, acyl, or amidyl; each of which is optionally substituted with R²⁷;
each R²⁵ and R²⁶ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, oxo, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R²⁸)₂, S(O)₂N(R²⁸)₂, phosphate, C₁-C₄ alkylenedioxy, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl;
R²⁷ is halo, hydroxy, carboxy, carboxylate, oxo, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R²⁸)₂, S(O)₂N(R²⁸)₂, phosphate, C₁-C₄ alkylenedioxy, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl;
R²⁸ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl;
Q is S. O, or NR²⁹;
R²⁹ is H, C₁-C₆ alkyl, C₇-C₁₂ aralkyl, or C₇-C₁₂ heteroaralkyl;
P is N or CR³⁰; and
R³⁰ is H or C₁-C₆ alkyl.

43. The method of claim 42, wherein R²¹ and R²², together with the carbons to which they are attached, form C₅-C₁₀ cycloalkenyl, C₅-C₁₀ heterocycloalkenyl, C₆-C₁₀ aryl, or C₅-C₁₀ heteroaryl.

44. The method of claim 43, wherein R²¹ and R²², together with the carbons to which they are attached, form C₅-C₁₀ cycloalkenyl.

45. The method of claim 42, wherein R²³ is hydroxy, C₁-C₁₀ alkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, C₁-C₆ dialkyl amino, or acyl.

46. The method of claim 45, wherein R²³ is C₃-C₈ cycloalkyl, C₅-C₈ heterocyclyl, C₅-C₁₀ cycloalkenyl, or C₅-C₁₀ heterocycloalkenyl.

47. The method of claim 42, wherein R²⁴ is halo, hydroxy, C₁-C₁₀ alkyl, C₁-C₆ haloalkyl, C₁-C₁₀ alkoxy, C₁-C₆ haloalkoxy, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, C₁-C₆ alkyl amino, C₁-C₆ dialkyl amino, mercapto, thioalkoxy, thioaryloxy, or thioheteroaryloxy.

48. The method of claim 47, wherein R²⁴ is C₁-C₁₀ alkyl, thioalkoxy, thioaryloxy, or thioheteroaryloxy.

49. The method of claim 48, wherein R²⁴ is C₁-C₁₀ alkyl or thioalkoxy; and R²⁷ is carboxy, carboxylate, cyano, nitro, amino, C₁-C₆ alkyl amino, C₁-C₆ dialkyl amino, SO₃H, sulfate, S(O)N(R²⁸)₂, S(O)₂N(R²⁸)₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl.

50. The method of claim 49, wherein R²⁴ is C₁-C₁₀ alkyl or thioalkoxy; substituted with carboxy, carboxylate, amidyl, or aminocarbonyl.

51. The method of claim 42, wherein X is S.

52. The method of claim 42, wherein Y is N.

53. The method of claim 42, wherein
R²¹ and R²², together with the carbons to which they are attached, form C₅-C₁₀ cycloalkenyl, C₅-C₁₀ heterocycloalkenyl, C₆-C₁₀ aryl, or C₅-C₁₀ heteroaryl;
R²³ is hydroxy, C₁-C₁₀ alkyl, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, C₁-C₆ dialkyl amino, or acyl;
R²⁴ is C₁-C₁₀ alkyl, thioalkoxy, thioaryloxy, or thioheteroaryloxy;
R²⁷ is carboxy, carboxylate, cyano, nitro, amino, C₁-C₆ alkyl amino, C₁-C₆ dialkyl amino, SO₃H, sulfate, S(O)N(R²⁸)₂, S(O)₂N(R²⁸)₂, phosphate, acyl, amidyl, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl or alkoxyaminocarbonyl;
Q is S; and
P is N.

54. The method of claim 42, wherein
R²¹ and R²², together with the carbons to which they are attached, form C₅-C₁₀ cycloalkenyl, or C₅-C₁₀ heterocycloalkenyl;
R²³ is C₁-C₁₀ alkyl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, amino, C₁-C₆ alkyl amino, or C₁-C₆ dialkyl amino;
R²⁴ is C₁-C₁₀ alkyl, thioalkoxy, thioaryloxy, or thioheteroaryloxy;
R²⁷ is carboxy, carboxylate, SO₃H, sulfate, S(O)N(R²⁸)₂, S(O)₂N(R²⁸)₂, phosphate, aminocarbonyl, C₁-C₆ alkyl aminocarbonyl, C₁-C₆ dialkyl aminocarbonyl, or C₁-C₁₀ alkoxycarbonyl;
Q is S; and
P is N.

55. A method for treating an HIV-mediated disorder in a subject, the method comprising administering to the subject an effective amount of a compound having a formula (IV): 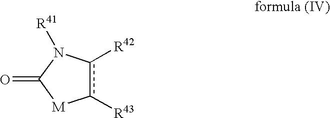
wherein;
R⁴¹ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, amino, C₁-C₆ alkyl amino, C₁-C₆ dialkyl amino, acyl, aminocarbonyl, C₁-C₆ alkyl aminocarbonyl, C₁-C₆ dialkyl aminocarbonyl, C₁-C₁₀ alkoxycarbonyl, or C₁-C₁₀ thioalkoxycarbonyl; each of which is optionally substituted with one or more R⁴⁴;
R⁴² and R⁴³, together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which is optionally substituted with 1-4 R⁴⁵; or
R⁴⁴ is H, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, C₆-C₁₀ aryloxy, C₅-C₁₀ heteroaryloxy, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R⁴⁶)₂, S(O)₂N(R⁴⁶)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, or hydroxyaminocarbonyl or alkoxyaminocarbonyl;
R⁴⁵ is halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, oxo, carboxy, carboxylate, cyano, nitro, amino, C₁-C₆ alkyl amino, C₁-C₆ dialkyl amino, mercapto, thioalkoxy, thioaryloxy, thioheteroaryloxy, SO₃H, sulfate, S(O)N(R⁴⁶)₂, S(O)₂N(R⁴⁶)₂, phosphate, C₁-C₄ alkylenedioxy, acyl, amido, aminocarbonyl, C₁-C₆ alkyl aminocarbonyl, C₁-C₆ dialkyl aminocarbonyl, C₁-C₁₀ alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl, or alkoxyaminocarbonyl;
R⁴⁶ is H, C₁-C₁₀ alkyl, C₆-C₁₀ aryl, C₅-C₁₀ heteroaryl, C₇-C₁₂ aralkyl, C₇-C₁₂ heteroaralkyl, C₂-C₁₂ alkenyl, C₂-C₁₂ alkynyl, or C₅-C₁₀ cycloalkenyl; and
M is NR⁴⁷, S, or O;
R⁴⁷ is H, halo, hydroxy, C₁-C₁₀ alkyl, C₁-C₆ haloalkyl, C₁-C₁₀ alkoxy, C₁-C₆ haloalkoxy, C₂-C₁₂ alkenyl, C₂-C₁₂ alkynyl, carboxy, carboxylate, amino, C₁-C₆ alkyl amino, C₁-C₆ dialkyl amino, acyl, aminocarbonyl, C₁-C₆ alkyl aminocarbonyl, C₁-C₆ dialkyl aminocarbonyl, or C₁-C₁₀ alkoxycarbonyl.

56. The method of claim 55, wherein R⁴² and R⁴³, together with the carbons to which they are attached, form C₆-C₁₀ aryl, or C₆-C₁₀ heteroaryl.

57. The method of claim 56, wherein R⁴² and R⁴³, together with the carbons to which they are attached, form phenyl.

58. The method of claim 57, wherein R⁴² and R⁴³, together with the carbons to which they are attached, form phenyl; and are substituted with halo or C₁-C₁₀ alkyl.

59. The method of claim 55, wherein R⁴¹ is C₁-C₁₀ alkyl; and R⁴⁴ is H, halo, C₆-C₁₀ aryl, C₅-C₁₀ heteroaryl, C₃-C₈ cycloalkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, acyl, amino, C₁-C₆ alkyl amino, C₁-C₆ dialkyl amino, amido, aminocarbonyl, C₁-C₆ alkyl aminocarbonyl, C₁-C₆ dialkyl aminocarbonyl, carboxy, or C₁-C₁₀ alkoxycarbonyl.

60. The method of claim 55, wherein M is O.

61. The method of claim 55, wherein
R⁴¹ is C₁-C₁₀ alkyl; and R⁴⁴ is acyl, amino, C₁-C₆ alkyl amino, C₁-C₆ dialkyl amino, amido, aminocarbonyl, C₁-C₆ alkyl aminocarbonyl, C₁-C₆ dialkyl aminocarbonyl, carboxy, or C₁-C₁₀ alkoxycarbonyl;
R⁴² and R⁴³, together with the carbons to which they are attached, form C₆-C₁₀ aryl, or C₆-C₁₀ heteroaryl; and
M is O.


SEQUENCE LISTING (9 sequences)

SEQ ID NO: 1 - protein, 747 aa, Homo sapiens
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 2 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 3 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 4 - protein, 314 aa, Homo sapiens
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 5 - protein, 310 aa, Homo sapiens
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 6 - protein, 355 aa, Homo sapiens
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 7 - protein, 400 aa, Homo sapiens
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 8 - protein, 101 aa, Human immunodeficiency virus 1
MEPVDPNLEPWNHPGSQPTTACSNCYCKVCCWHCQLCFMTKGLSISYGRKKRKRRRGTPHGSEDHQNLISKQPSSQPRGDPTGPKEQKKKVESKAEADPFD

SEQ ID NO: 9 - protein, 133 aa, Human immunodeficiency virus 2
MGIPLQEQENSLEFSSERSSSTSEEGANTRGLDNQGEEILSQLYRPLEACRNKCYCKKCCYHCQLCFLKKGLGICYDHSRKRSSKRAKVTAPTASNDLSTRARDGQPAKKQKKEVETTRTTDPGLGRSDTSTS